SEQUENCE LISTING (26 sequences)

SEQ ID NO: 1 - DNA, 14 bp, Artificial
  synthetic
tatttgacaacgcg

SEQ ID NO: 2 - DNA, 17 bp, Artificial
  synthetic
ttttaaaccgcattggg

SEQ ID NO: 3 - DNA, 24 bp, Artificial
  synthetic
gaggttaacctaagcactgccaag

SEQ ID NO: 4 - DNA, 43 bp, Artificial
  synthetic
catagagtatgcagatatcgttagtgttacaggtttagttttg

SEQ ID NO: 5 - DNA, 42 bp, Artificial
  synthetic
gtaacactaacgatatctgcatactctatgtcattttcatgg

SEQ ID NO: 6 - DNA, 26 bp, Artificial
  synthetic
cagcgacatgaacttaagtgagctgc

SEQ ID NO: 7 - DNA, 65 bp, Artificial
  synthetic
cacactaaacggtacacaggaaacaggagacacaacttgtgactgccgcggagatgtttctgccc

SEQ ID NO: 8 - DNA, 66 bp, Artificial
  synthetic
gggcagaaacagtctccgcggcagtcacaagttgtgtctcctgtttcctgtgtaccgtttagtgtg

SEQ ID NO: 9 - DNA, 20 bp, Artificial
  synthetic
cttaaggactagtttcgcgc

SEQ ID NO: 10 - DNA, 22 bp, Artificial
  synthetic
caagctagctcgaggaatcatg

SEQ ID NO: 11 - DNA, 20 bp, Artificial
  synthetic
cttaaggactagtttcgcgc

SEQ ID NO: 12 - DNA, 27 bp, Artificial
  synthetic
cacgctagcaagttaaccatgtcttgg

SEQ ID NO: 13 - DNA, 21 bp, Artificial
  synthetic
gaaaccgaattctcttggaac

SEQ ID NO: 14 - DNA, 24 bp, Artificial
  synthetic
gaattctagctagctcagctatag

SEQ ID NO: 15 - DNA, 78 bp, Artificial
  synthetic
tcgagctccgcatttggcgggcgggattggtcttcgtagaacctaatctcgtgggcgtggtagtcctcaggtacaaat

SEQ ID NO: 16 - DNA, 79 bp, Artificial
  synthetic
agcttatttgtacctgaggactaccacgcccacgagattaggttctacgaagaccaatcccgcccgccaaatgcggagc

SEQ ID NO: 17 - DNA, 18 bp, Artificial
  synthetic
atggccgccagtcttttg

SEQ ID NO: 18 - DNA, 20 bp, Artificial
  synthetic
ttatggcctggggcgtttac

SEQ ID NO: 19 - DNA, 24 bp, Artificial
  synthetic
tccggttattttccaccatattgc

SEQ ID NO: 20 - DNA, 22 bp, Artificial
  synthetic
ttatcatcgtgtttttcaaagg

SEQ ID NO: 21 - DNA, 19 bp, Artificial
  synthetic
atgagcaccaactcgtttg

SEQ ID NO: 22 - DNA, 21 bp, Artificial
  synthetic
gttttaaaccgcattgggagg

SEQ ID NO: 23 - DNA, 23 bp, Artificial
  synthetic
gtgtctagagaatgcaatagtag

SEQ ID NO: 24 - DNA, 20 bp, Artificial
  synthetic
gtcaaagaatccaattgtgc

SEQ ID NO: 25 - DNA, 20 bp, Artificial
  synthetic
atgactacgtccggcgttcc

SEQ ID NO: 26 - DNA, 18 bp, Artificial
  synthetic
ctacatgggggtagagtc

(19) United States
(12) Patent Application Publication
(10) Pub. No.: US 2009/0232800 A1
(43) Pub. Date: Sep. 17, 2009

(54) E1-MINUS ADENOVIRUSES AND USE THEREOF

(76) Inventor: Per Sonne Holm, Fuestenfeldbruck (DE)

Correspondence Address: DYKEMA GOSSETT PLLC, 10 S. WACKER DR., STE. 2300, CHICAGO, IL, 60606, US

(21) Appl. No.: 11/813,089

(22) PCT Filed: Jan. 2, 2006
(86) PCT No.: PCT/EP2006/000010

§ 371 Date: Aug. 5, 2008

(30) Foreign Application Priority Data
Dec. 31, 2004 (DE) 10 2004 063 662.1

(51) Int. Cl. A61K 39/395; A61K 35/76; A61K 38/00; C12Q 1/68; G01N 33/53; C12Q 1/02; C12N 15/00; C12N 7/01; C12N 15/63; A61K 31/7088; C07H 21/04

(52) U.S. Cl. 424/133.1; 424/93.2; 424/94.5; 435/6; 435/7.1; 435/29; 435/455; 435/235.1; 435/320.1; 514/44 R; 536/23.72

(57) ABSTRACT

The present invention is related to a virus, preferably an adenovirus, characterised in that the virus comprises:
a lacking functional wildtype E1 region, and
a transporter for the transport of YB-1 into the nucleus of a cell which is infected with the virus.

The invention relates to E1-minus adenoviruses and nucleic acids coding therefore and the use thereof.

A number of therapeutic concepts are currently used in the treatment of tumors. Apart from using surgery, chemotherapy and radiotherapy are predominant. All these techniques are, however, associated with considerable side effects for the patient. The use of replication selective oncolytic viruses provides for a new platform for the treatment of tumors. In connection therewith a selective intratumoral replication of a virus is initiated which results in virus replication, lysis of the infected tumor cell and spreading of the virus to adjacent tumor cells. As the replication capabilities of the virus are limited to tumor cells, normal tissue is spared from replication and thus from lysis by the virus.

For the time being, several viral systems are subject to clinical trials aiming at tumor lysis. One example for such an adenovirus is d11520 (Onyx-015) which has been successfully used in clinical phases I and II (Khuri, F. et al. Nature Medicine 6, 879-885, 2000). Onyx-015 is an adenovirus having a completely deleted E1B-55kDa gene. The complete deletion of the E1B-55kDa protein of the adenovirus is based on the discovery that replication and thus lysis of cells is possible with an adenoviral vector whereby the cells have a p53 deficiency (Kirn, D. et al., Proc. Am. Soc. Clin. Oncol. 17, 391a, 1998), whereby normal cells are not harmed. More particularly, the E1B-55kDa gene product is involved in the inhibition of p53, the transport of viral mRNA and the switching off of the protein synthesis of the host cell. The inhibition of p53 occurs via formation of a complex consisting of p53 and the adenoviral encoded E1B-55kDa protein and/or a complex consisting of E1B-55kDa and E4orf6. p53 coded by TP53 is the starting point for a complex regulatory mechanism (Zambetti, G. P. et al., FASEB J. 7, 855-865, 1993), which results, among others, in an efficient suppression of the cellular replication of viruses like adenoviruses. The gene TP 53 is deleted or mutated in about 50% of all human tumors which results in the absence of a—desired—apoptosis due to chemotherapy or radiation therapy and thus in an usually unsuccessful tumor treatment.

A further concept of tumorlytic adenoviruses is based on the discovery that if the E1A protein is present in a specifically deleted form or comprises one or several mutations, which do not affect the binding of Rb/E2F and/or p107/E2F and/or p130/E2F, such adenovirus will not induce the entry of the infected cells into the S phase and will be capable of replicating in tumor cells which do not have a functional Rb protein. Additionally, the E1A protein can be deleted at the N-terminus and can comprise one or several mutations in the region of amino acid positions 1 to 76 of the E1A proteins, respectively, in order to inhibit the binding of E1A to p300 and thus to provide for a more selective replication in tumor cells. These approaches are described in an exemplary manner in European patent EP 0 931 830. Examples for such viruses are AdΔ24, dl922-947, E1Ad/01/07 and CB016 (Howe, J. A. et al., Molecular Therapy 2, 485-495, 2000; Fueyo, J. et al., Oncogene 19, 2-12, 2000; Heise, C. et al., Nature Medicine 6, 11341139, 2001; Balague, C. et al., J. Virol. 75, 7602-7611, 2001). These adenoviral systems for oncolysis known in the prior art thus comprise distinct deletions in the E1A protein, whereby such deletions had been made under the assumption that a functional Rb protein and a complex consisting of intact Rb protein and E2F, respectively, would block an efficient in vivo replication and in order to provide an adenoviral replication in vivo in Rb-negative/mutated cells only. These adenoviral systems according to the prior art are based on E1A in order to control in vivo replication by means of the early E2 promoter (E2 early promoter) and free E2F (Dyson, N. Genes & Development, 12, 2245-2262, 1998).

Another form of tumorlytic adenoviral systems is based on the use of selective promoters for specifically expressing the viral oncogene E1A which provides for a selective replication in tumor cells (Rodriguez, R. et al., Cancer Res. 57, 2559-2563, 1997).

As described above, the selection of a cellular background which is appropriate for the mode of action underlying the respective concept, is important for the various concepts of adenoviral tumorlytic viruses. In other words, the various adenoviral systems currently known may only be used if distinct molecular biological prerequisites are realized. This limits the use of such systems to distinct patient groups.

A particular problem in the treatment of tumor diseases arises once the patients develop a so-called multidrug resistance (MDR) which represents a particularly well studied form of resistance of tumors against cytostatics (Gottesman and Pastan, Annu. Rev. Biochem. 62, 385-427, 1993). It is based on the overexpression of the membrane-bound transport protein P-glycoprotein which belongs to the so-called ABC transporters (Stein, U. et al., JBC 276, 28562-69, 2001, J. Wijnholds, Novartis Found Symp., 243, 69-79, 2002). Bargou, R. C. et al. and Oda, Y. et al (Bargou, R. C. et al., Nature Medicine 3, 447-450, 1997; Clin. Cancer Res. 4, 2273-2277, 1998) were able to show that nuclear localisation of the human transcription factor YB-1 is directly involved in the activation of the expression of the P-glycoprotein. Further studies confirmed that YB-1 is transported into the nucleus by various stress conditions such as for example UV irradiation, administration of cytostatics (Koike, K. et al., FEBS Lett 17, 390-394, 1997) and hyperthermia (Stein, U. et al., JBC 276, 28562-69, 2001). Further studies confirmed that the nuclear localisation of YB-1 has an impact on another ABC transporter. This ABC transporter is referred to as MRP (multidrug resistance-related protein) and is involved in the formation of the so-called atypical, non-P-glycoprotein dependent multidrug resistance (Stein, U. et al., JBC 276, 28562-69, 2001).

The problem underlying the present invention is to provide a technical teaching and in particular a means which allows specifically to treat an organism, more particularly a human organism and a group of patients, respectively, with tumorlytically active agents. It is a further problem underlying the present invention to provide a means which is suitable to cause tumor lysis in patients suffering from tumor diseases which are resistant to cytostatics, particularly those which have a multidrug resistance. A further problem underlying the present invention is to provide for an adenovirus which is suitable for cell lysis. Another problem underlying the present invention was to provide a virus which replicates in tumor cells and particularly in tumor cells which have YB-1 in the nucleus independent of the cell cycle, or tumor cells with deregulated YB-1, and which shows a particularly high particle formation.

In accordance with the invention, the problem is solved by the subject matter of the attached independent claims. Preferred embodiments may be taken from the also attached dependent claims.

In a first aspect the problem is also solved in accordance with the present invention by a virus, preferably an adenovirus, whereby the virus comprises:
a lacking functional wildtype E1 region, and
a transporter for the transport of YB-1 into the nucleus of a cell which is infected with the virus.

In an embodiment of the first aspect the virus comprises a nucleic acid coding for protein IX and expresses protein IX.

In an embodiment of the first aspect the lacking functional wildtype E1A region is E1A-minus.

In an embodiment of the first aspect the lacking functional wildtype E1 region is E1B-minus.

In a preferred embodiment of the first aspect the lacking wildtype E1 region is E1B55K-minus and/or E1B19K-minus and/or protein IX-minus.

In an embodiment of the first aspect the transporter is a transporter provided by the virus.

In a preferred embodiment of the first aspect the transporter is a viral transporter.

In an embodiment of the first aspect the transporter comprises protein E4orf6.

In an embodiment of the first aspect the transporter comprises protein E1B55K.

In an embodiment of the first aspect the transporter comprises a complex of E4orf4 and E1B55K.

In an embodiment of the first aspect the transporter is coded by a nucleic acid, whereby the nucleic acid is under the control of a promoter.

In a preferred embodiment of the first aspect the transporter is a complex of at least two factors and whereby each factor is coded by a nucleic acid, whereby both nucleic acids are controlled by a shared promoter.

In a preferred embodiment of the first aspect both nucleic acid are connected through an element which controls the expression strength, whereby the element is preferably selected from the group comprising IRES.

In a preferred alternative embodiment of the first aspect the transporter is a complex of at least two factors and whereby each factor is coded by a nucleic acid, whereby both nucleic acids are controlled by a proprietary promoter.

In an embodiment of the first aspect the promoter is different from the E4 promoter, in particular the adenoviral E4 promoter, and is different from the E1B promoter, in particular the adenoviral E1B promoter.

In an embodiment of the first aspect the promoter is selected from the group comprising tissue-specific promoters, tumor-specific promoters, the CMV-promoter, viral promoters and particularly adenoviral promoters, under the proviso that these are different from the E4 promoter, the E1B promoter and preferably also different from the E2-late promoter.

In an embodiment of the first aspect the nucleic acid coding for the transporter has a 3′-UTR at the 3′ end of E1B55K.

In an embodiment of the first aspect the nucleic acid coding for the transporter does not comprise an E1B55K coding nucleic acid if the lacking wildtype E1 region is E1B55K-positive.

In an embodiment of the first aspect the nucleic acid coding for the transporter codes for E1B55K and E1B19K.

In an embodiment of the first aspect the nucleic acid coding for the transporter codes for protein IX.

In a preferred embodiment of the first aspect the nucleic acid coding for the E1B55K and E1B19K is under the control of a promoter.

In an alternative preferred embodiment of the first aspect the nucleic acid coding for the E1B55K and/or E1B19K and/or protein IX is under the control of a promoter, whereby the promoter is different from an E1A-dependent promoter.

In an embodiment of the first aspect the lacking functional wildtype E1 region is E1A13S-minus and/or E1A12S-minus.

In an embodiment of the first aspect the lacking functional wildtype E1 region is E1A13S-minus.

In an embodiment of the first aspect preferably the lacking wildtype E1 region is E1A13S-minus and E1A12-minus, whereby the virus comprises a nucleic acid coding for the E1A12S protein, whereby the nucleic acid is preferably a heterologous nucleic acid.

In a preferred embodiment of the first aspect the nucleic acid coding for the E1A12S protein is under the control of a promoter, whereby the promoter is preferably a YB-1 dependent promoter and more preferably selected from the group comprising the adenoviral E2-late promoter, the MDR-promoter and the DNA polymerase-alpha promoter.

In an embodiment of the first aspect and in particular the preceding embodiment the nucleic acid(s) coding for the transporter code for E4orf6 and E1B55K.

In an embodiment of the first aspect and in particular the preceding embodiment the virus comprises a nucleic acid coding for protein IX, whereby preferably the nucleic acid coding for E1A12S and the nucleic acid coding for protein IX are under the control of a shared promoter, whereby more preferably both nucleic acids are linked to each other through an expression regulating element, whereby the element is more preferably selected from the group comprising IRES.

In an embodiment of the first aspect the nucleic acid coding for the E1A12S protein and the nucleic acid coding for the protein IX are each under the control of a promoter, whereby the promoter is preferably the same promoter.

In a preferred embodiment of the first aspect the promoter is a YB-1 dependent promoter, which is preferably selected from the group comprising the adenoviral E2-late promoter, the MDR promoter and the DNA polymerase-alpha promoter.

In an embodiment of the first aspect the virus comprises a YB-1 coding nucleic acid.

In a preferred embodiment of the first aspect and in particular the preceding embodiment the nucleic acid coding for the E1A12S protein and the nucleic acid coding for the YB-1 are under the control of a shared promoter, whereby preferably both nucleic acids are linked to each other by an expression regulating element, whereby the element is preferably selected from the group comprising IRES.

In a preferred alternative embodiment of the first aspect the nucleic acid coding for YB-1 and the nucleic acid coding for E1A12S protein are each under the control of a promoter, whereby the promoter is preferably the same promoter.

In a preferred embodiment of the first aspect the promoter is a YB-1 dependent promoter which is preferably selected from the group comprising the adenoviral E2-late promoter, the MDR promoter and the DNA polymerase-alpha promoter.

In an embodiment of the first aspect the nucleic acid coding for E1A12S is cloned into the E3 region or E4 region.

In an embodiment of the first aspect the nucleic acid coding for E1A12S and the nucleic acid coding for the protein IX or the nucleic acid coding for the YB-1 are cloned into the E3 region or the E4 region.

In an embodiment of the first aspect the expression of the nucleic acid coding for protein IX is controlled by a promoter different from E1B via E1B19K or via E12AS.

In an embodiment of the first aspect the virus comprises at least one transgene which is preferably cloned into the E3 region.

In a preferred embodiment of the first aspect the virus comprises at least one transgene which is preferably cloned into the E4 region.

In an embodiment of the first aspect the virus comprises a nucleic acid coding for the RGD motif, whereby the RGD motif is preferably cloned into the HI-loop domain of the fibre knob.

In an embodiment of the first aspect the virus further comprises MLP genes and/or E2A genes and E2B genes and/or E3 genes and/or E4 genes.

In an embodiment of the first aspect the virus is replication deficient in cells which do not contain YB-1 in the nucleus.

In an embodiment of the first aspect the virus can replicate in cells which have YB-1 in the nucleus, in particular have YB-1 in the nucleus independent of the cell cycle.

In an alternative embodiment of the first aspect the virus is replication deficient in cells where or in which YB-1 is deregulated.

In a further alternative embodiment of the first aspect the virus is capable of replicating in tumor cells, preferably tumor cells which are resistant against cytostatics and/or radiation.

In a preferred embodiment of the first aspect the cells are multiple-drug or multidrug resistant.

In a second aspect the problem is solved in accordance with the present invention by a nucleic acid coding for a virus according to the first aspect of invention.

In a third aspect the problem is solved in accordance with the present invention by the use of a virus according to the first aspect or a nucleic acid according to the second aspect or a vector comprising the same or a replication system comprising such nucleic acid or a part thereof, for the manufacture of a medicament.

In a fourth aspect the problem is solved in accordance with the present invention by the use of a virus according to the first aspect or a nucleic acid according to the second aspect for replication in cells, whereby the cells contain YB-1 in the nucleus, preferably contain YB-1 in the nucleus independent of the cell cycle, or the cells contain deregulated YB-1 or the cells are tumor cells, preferably tumor cells which are resistant against cytostatics and/or radiation.

In an embodiment of the fourth aspect the cells contain YB-1 in the nucleus after or due to a measure which is applied to the cell or has been applied to the cell and is selected from the group comprising radiation, application of cytostatics and hyperthermia.

In an embodiment of the third aspect the medicament is for the treatment of tumors and/or cancer(s) and/or for the restoration of sensitivity of cells to cytostatics and/or radiation, whereby preferably the cells are tumor cells which are resistant against cytostatics and/or radiation.

In an embodiment of the third aspect at least one part of the cells forming the tumor are cells which have YB-1 in the nucleus, preferably contain YB-1 in the nucleus independent of the cell cycle, or at least one part of the cells forming the tumor have deregulated YB-1 or at least one part of the cells forming the tumor are tumor cells, more preferably tumor cells which are resistant against cytostatics and/or radiation.

In a preferred embodiment of the third aspect the cells, particularly the cells forming the tumor or parts thereof, are resistant, in particular multi-resistant against drugs, preferably antitumor agents and more preferably cytostatics.

In an embodiment of the third and fourth aspect the cells show an expression, more preferably an overexpression of the membrane bound transport protein P-glycoprotein.

In an embodiment of the third and fourth aspect the cells have YB-1 in the nucleus, and particularly the cells forming the tumor or part thereof have YB-1 in the nucleus.

In an embodiment of the third and fourth aspect the tumor contains YB-1 in the nucleus after induction of the transport of YB-1 into the nucleus.

In a preferred embodiment of the third and fourth aspect the transport of YB-1 into the nucleus is triggered by at least one measure which is selected from the group comprising radiation, application of cytostatics and hyperthermia.

In a preferred embodiment of the third and fourth aspect the measure is applied to a cell, an organ or an organism.

In a fifth aspect the problem is solved according to the invention by the use of a virus replication system, particularly an adenoviral replication system, comprising a nucleic acid which codes for a virus, particularly an adenovirus, according to the first aspect or a part thereof, and comprising a nucleic acid of a helper virus, whereby the nucleic acid of the helper virus comprises a nucleic acid sequence which codes for YB-1, and optionally complements the virus, preferably for the manufacture of a medicament, more preferably for the treatment of tumors and/or cancer(s) and/or for restoration of the sensitivity of cells to cytostatics and/or radiation, whereby the cells are preferably tumor cells which are resistant against cytostatics and/or radiation.

In an embodiment of the fifth aspect the viral nucleic acid, preferably the adenoviral nucleic acid and/or the nucleic acid of the helper virus are present as a replicable vector.

In a sixth aspect the problem is solved according to the invention by the use of a nucleic acid coding for a virus, preferably an adenovirus according to the first aspect for the manufacture of a medicament, preferably for the manufacture of a medicament for the treatment of tumors and/or for restoration of sensitivity of cells to cytostatics and/or radiation, whereby the cells are preferably tumor cells which are resistant against cytostatics and/or radiation.

In an embodiment of the sixth aspect the cells, and particularly the cells forming the tumor or parts thereof, are resistant, in particular multiple-resistant against drugs, preferably antitumor agents and more preferably cytostatics.

In a seventh aspect the problem is solved according to the invention by a vector comprising a nucleic acid according to the second aspect, preferably for the use according to the third and fourth aspect.

In eighth aspect the problem is solved according to the invention by the use of an agent interacting with YB-1 for the characterisation of cells, cells of a tumor tissue or patients, in order to determine whether such cells, cells of a tumor tissue or patients can/should be contacted and/or treated with a virus, in particular an adenovirus, according to the first aspect or a nucleic acid according to the second aspect.

In an embodiment of the eighth aspect the agent is selected from the group comprising antibodies, high affinity binding peptides, antikalines, aptamers, aptazymes and spiegelmers.

In a ninth aspect the problem is solved according to the invention by a pharmaceutical composition comprising a virus according to the first aspect, or a nucleic acid according to the second aspect or a viral replication system as described in the fifth aspect.

In an embodiment of the ninth aspect the composition comprises at least one further pharmaceutically active agent.

In a preferred embodiment of the ninth aspect the pharmaceutically active agent is selected from the group comprising cytokines, metalloproteinase inhibitors, angiogenesis inhibitors, cytostatics, cell cycle inhibitors, proteosome inhibitors, recombinant antibodies, inhibitors of the signal transduction cascade and protein kinases.

In an embodiment of the ninth aspect the composition comprises a combination of at least two compounds, whereby preferably any compound is each and independently selected from the group comprising cytostatics.

In a preferred embodiment of the ninth aspect at least two of the compounds target different target molecules.

In an embodiment of the ninth aspect at least two of the compounds are active through different modes of action.

In an embodiment of the ninth aspect at least one compound increases the infectability of a cell in which the virus is replicating.

In an embodiment of the ninth aspect at least one compound influences the availability of a compound in the cell, preferably increases the availability of the compound, whereby the compound mediates the uptake of the virus in one or the cell, preferably the one in which the virus replicates.

In an embodiment of the ninth aspect at least one of the compound mediates the transport of YB-1 into the nucleus, preferably increases the same.

In an embodiment of the ninth aspect at least one compound is a histone deacylase inhibitor.

In a preferred embodiment of the ninth aspect the histon deacylase inhibitor is selected from the group comprising trichostatine A, FR 901228, MS-27-275, NVP-LAQ824, PXD101 apicidine and scriptaid.

In an embodiment of the ninth aspect at least one compound is selected from the group comprising trichostatine A, FR 901228, MS-27-275, NVP-LAQ824, PXD101 apicidine and scriptaid.

In an embodiment of the ninth aspect at least one compound is a topoisomerase inhibitor.

In a preferred embodiment of the ninth aspect the topoisomerase inhibitor is selected from the group comprising camptothecin, irinotecan, toptecan, DX-895If, SN-38, 9-aminocamptothecin, 9-nitrocamptothecin, daunorubicin and etoposide.

In an embodiment of the ninth aspect the composition comprises trichostatine A and irinotecan.

In an embodiment of the ninth aspect the virus, in particular the virus according to the first aspect of the present invention is separated from one or both or all of the at least two compounds.

In a preferred embodiment of the ninth aspect at least one unit dose of the virus is separated from at least one unit dose of the or all further pharmaceutically active compound(s) or from one or the at least two compounds.

In a tenth aspect the problem is solved according to the invention by a kit comprising a virus, preferably a virus according to the first aspect of the present invention, and at least two pharmaceutically active agents, whereby each pharmaceutically active agent is individually and independently selected from the group comprising cytostatics.

The present invention is based on the surprising finding that the viruses according to the invention, i.e. viruses which lack a functional E1 region as present in the wildtype adenovirus, and which, at the same time, comprise a transporter and in particular code for a transporter which is capable of transporting or translocating YB-1 into the nucleus, is capable of replicating in cells which contain YB-1 in the nucleus independent of the cell cycle, or in cells which have deregulated YB-1.

Additionally the present inventor has found that the viruses according to the invention may also replicate independently of E1A13S, in particular if the replication is mediated by YB-1. In connection therewith, the replication occurs in particular in those cells as described above. As used herein, cells which contain YB-1 in the nucleus, preferably contain YB-1 in the nucleus independent of the cell cycle, also comprise those which contain YB-1 in the nucleus due to the use of the viruses according to the invention and in particular due to the infection of the cells with them.

Finally, the present inventor has found that protein IX is an important factor, in particular for the effectiveness of the viruses according to the invention when used as oncolytic viruses and that this factor is expressed by the constructs disclosed herein which results in a high particle formation also in YB-1 mediated E1A13S independent viral replication.

Cells which contain YB-1 in a deregulated form are those which have at least one of the following characteristics and/or those which contain YB-1, whereby YB-1 exhibits at least one of the following characteristics: (1) YB-1 is overexpressed in the cells, preferably overexpressed independent of the cell cycle, whereby preferably as a measure for the expression it is referred to the expression of YB-1 in normal cells, i.e. cells which are different from tumor cells or cells and cell lines, respectively such as the following ones: hepatocytes as well as fibroblast cell lines WI38 and CCD32-Lu. Preferably an overexpression is an expression which is increased by the factor of about 2 to 10, preferably 5 to 10. Methods for measuring the expression and in particular the overexpression are known to the persons skilled in the art and comprise, among others, the measuring the protein concentration, in particular of YB-1, measuring the RNA, in particular RNA of YB-1, Western Blot Analysis, Northern Blot Analysis and RT-PCR, each preferably related to or of YB-1. Rather than YB-1 also surrogate markers as described herein may be used. Examples for cell lines which show an overexpression of YB-1 are the following ones: colon carcinoma cell line 257RDB, pancreas carcinoma cell line 181RDB, mamma carcinoma cell line MCF-7Adr, prostate carcinoma cell line DU145, prostate carcinoma cell line PC3, glioma cell line U373, glioma cell line U87, lung carcinoma cell line A549, liver carcinoma cell lines Hep3B and HepG2. YB-1 which is present in the cell, allows the replication of the viruses according to the invention. It is preferred within the present invention that the replication efficacy under such conditions is different from a strongly reduced replication.

As used herein in an embodiment the term functional wildtype E1 region refers in particular to an E1 region as contained in the wildtype adenovirus Ad5. In an embodiment the term lacking functional wildtype E1 region refers to an E1 region which does not contain or not completely contain one or several of the functions and functionalities contained in adenoviruses of the wildtype. Functionality or function, generally referred to herein in the following as function, is represented or mediated by a nucleic acid or a protein, preferably a protein.

In connection with the present invention the lack of the function may be caused by the function not being active at the translation level, i.e. that the protein mediating the function is not present, although the nucleic acid coding therefore is still present in the viral genome. This may, for example, be achieved by the translation controlling regulatory elements not being active, preferably not being in the regulatory and controlling context as present in viruses of the wildtype for the respective feature, whereby such regulatory elements can, for example, be present at the 3′UTR of the mRNA, which, among others, provides for the stability of the mRNA.

In connection with the present invention the lack of the function can alternatively or additionally be caused by the function not being active at the transcription level, i.e. the function mediating protein is not present and that the nucleic acid coding therefore is not contained or not completely contained in the viral genome. It is within this embodiment that the nucleic acid contains one or several mutations which result in the loss of function. Such mutations are preferably point mutations and/or deletions comprising several bases and/or a complete deletion of the open reading frame or of the nucleic acid coding for the protein.

A function is lacking in the sense of the above if the protein does not exhibit all of the functions or activities of the corresponding protein of the wildtype. In an embodiment the measure for the activity is the extent of replication which is achieved under such conditions, whereby it is preferably different from a strongly different replication.

In a preferred embodiment of the present invention the function is also lacking when the function is, compared to the wildtype virus, contained in the virus in a different regulatory context. A different regulatory context is in an embodiment one, whereby the function is expressed, compared to other functions, at a different point in time and/or is under the control of a different element which controls or influences transcription and/or translation. Such an element is in a particular embodiment the promoter.

The lack of a function in the above sense is also indicated herein by referring to the respective function as “minus”. For example, the lack of E1A13S is referred to as E1A13S-minus.

In an embodiment a strongly reduced replication is in particular a replication which is reduced, compared to the wildtype, by a factor of 2, preferably by a factor of 5, more preferably by a factor of 10 and most preferably by a factor of 100. In a preferred embodiment the comparison of the replication is performed using identical or similar cell lines, identical or similar virus titers for the infection (multiplicity of infection, MOI, or plaque forming unit, pfu) and/or identical or similar assay conditions. Replication means in particular particle formation. In further embodiments the measure for replication can be the extent of viral nucleic acid synthesis. Methods for determining the extent of the synthesis of viral nucleic acids are known to the persons skilled in the art as are methods for the determination of particle formation.

The viruses according to the present invention comprise a transporter for the transport of YB-1 into the nucleus. In a preferred embodiment the transporter is a protein, preferably a viral protein. YB-1 which is transported by the transporter into the nucleus of the cell, is one which is preferably a deregulated YB-1, in particular as defined herein. However, it is also within the present invention that YB-1 is a YB-1 which, alternatively or in addition to deregulated YB-1, is encoded by the virus of the invention and is expressed by said virus in the cell which is infected by said virus.

The cells in which the viruses of the invention transport YB-1 into the nucleus, are preferably those which contain deregulated YB-1.

It is within the skills of the persons of the art to determine whether a virus comprises such a transporter or is coding therefore. In an embodiment a cell may be used whereby such cell does not contain YB-1 in a cell cycle independent manner in the nucleus such as the cervix carcinoma cell line HeLa or the osteosarcoma cell line U2OS, and it can subsequently be determined whether due to the infection and the subsequent replication of the virus the thus infected cell contains YB-1 in the nucleus. In an alternative embodiment the cell used is a cell which contains deregulated YB-1. The detection of YB-1 in the nucleus under such experimental conditions may be performed by a person skilled in the art by using the means described herein, in particular by using an antibody directed against YB-1. If, under the influence of the virus, YB-1 is detected in the nucleus, the tested virus comprises a or the transporter.

It is within the present invention that the E1 region, with regard to one or both protein groups which are encoded in the E1 region, is “minus” in the sense of the above. The two protein groups are the group of the E1A proteins, in particular the E1A 13S protein, also referred to herein as E1A 13S, and the E1A12S protein, also referred to herein as E1A12S, and the group of the E1B proteins, in particular the E1B55K protein, also referred to herein as E1B55K, the E1B19K protein, also referred to herein as E1B19K, and protein IX.

It is within an embodiment of the present invention that the virus is E1A13S minus, if it is under the control of a promoter which is different from the E1A promoter, preferably the adenoviral E1A promoter and more preferably the adenoviral E1A promoter of the wildtype; that the virus is E1A12S-minus if it is under the control of a promoter which is different from the E1A promoter, preferably the adenoviral E1A promoter and more preferably the adenoviral E1A promoter of the wildtype; that the virus is E1B55K-minus, if it is under the control of a promoter which is different from the E1B promoter, preferably the adenoviral E1A promoter and more preferably the adenoviral E1B promoter of the wildtype; that the virus is E1B19K-minus, if it is under the control of a promoter which is different from the E1B promoter, preferably the adenoviral E1B promoter and more preferably the adenoviral E1B promoter of the wildtype; and that it is protein IX-minus if it is under the control of a promoter which is different from the E1BIX promoter, preferably the adenoviral E1BIX promoter and more preferably the adenoviral E1BIX promoter of the wildtype and, while it is under the control of the E1BIX promoter, the promoter is inactive due to lack of viral factors in particular, which control the activity of the E1BIX promoter; the latter is thus an example that the regulatory context has been changed, more specifically that the regulatory context is indirectly changed or changed at a higher integration or regulatory level. In general, the term changed regulatory context also comprises changes which are active indirectly or at a higher integration or regulatory level, however, in any case are different from the circumstances of the wildtype, in particular of the wildtype adenovirus.

In an embodiment of the present invention the virus is E1A13S-minus. In a further embodiment the virus is also E1A12-minus. It is particularly preferred if the viral E1A12S is under the control of a promoter the activity of which is controlled by YB-1, in particular is activated by YB-1. These promoters are referred to herein also as YB-1-dependent promoters. A particularly preferred YB-1 dependent promoter is the adenoviral E2-late promoter. By this construction it is ensured that E1A12S is activated in viral replication only once YB-1 is contained in the nucleus. This is achieved in case of cells having deregulated YB-1 by the transporter of the virus according to the present invention which translocates the deregulated YB-1 into the nucleus of the infected cell. Due to the, compared to the expression in the wildtype, chronologically reversed expression of the viral transporters and of E1A12S the specificity of the expression of E1A12S is ensured in only those cells which contain YB-1 in deregulated form and thus the replication of the virus and, consequently, lysis is limited to these very cells which is, from a safety point of view, an essential advantage of this construction of the viruses.

Having this in mind the particle number was to be increased in the YB-1 dependent replication. The present inventor has recognised that also in YB-1 dependent replication protein IX plays an important role and that its expression remains unchanged by the aforedescribed chronological change of the expression of the transporter which is preferably provided by the proteins of the E1B region, and that E1A12S is not affected if the constructs disclosed herein are realised. The adenoviral constructs described in the prior art for the YB-1 dependent replication showed despite outstanding oncolytical characteristics a particle formation which is low for some applications which, for example, requires another application of the oncolytic virus. Such another application of viruses is, in principle, possible, however, not desired in most cases. With the constructs described herein, particle formation could be significantly increased. Insofar the present invention is related to the use of protein IX and/or a nucleic acid coding therefore, for the formation of viral, in particular adenoviral particles in connection with YB-1 dependent replicating viruses, in particular adenoviruses. Furthermore, the present invention is related to the use of viruses, preferably adenoviruses, which are replicating in a YB-1 dependent manner for the manufacture of a medicament, whereby the viruses comprise protein IX and/or a nucleic acid coding therefor. In a particularly preferred embodiment the medicament is for the treatment of tumors and tumor diseases as described herein. In a further, additional or alternative embodiment the medicament is for reversing a resistance in animal cells as described herein and/or for the restoration of the sensitivity of the cells towards cytostatics and/or radiation, whereby the cells are preferably tumor cells which show a resistance, particularly one as described herein, preferably a resistance against cytostatics and radiation, as in particular described herein.

It is within the present invention that the various transgenes are cloned at appropriate sites within the viral genome. Particularly preferred are the regions E1, E2A, E2B, E3 and E4. The cloning of the transporters into the E1 region is particularly preferred. It will be appreciated by the persons skilled in the art that the cloning of these transgenes into said sites of the viral genome can partially or completely inactivate or delete the genes coded by such sites. It is, however, also within the present invention that the genes coded by the respective site may partially or completely remain active.

The viruses of the invention are preferable adenoviruses.

The term treatment of a disease or condition comprises in a preferred embodiment also prevention of such disease or condition.

The adenoviral protein IX is cementing the capsid structure and is essential for the packaging of viral DNA in virions (Boulanger et al., Journal of Virology, 44, 783-800, 1979; Jones and Shenk, Cell, 17, 683-689, 1979). The gene is located in the viral genome between positions 3581 and 4071 (Colby and Shenk T, Journal of Virology, 1981, 39, 977-980), whereby the gene for protein IX is expressed only starting from replicating DNA molecules (Matsui T et al., Molecular and Cellular Biology, 1986, 6, 4149-4154).

The virus Xvir03-3′UTR, which is as such described in the prior art and is replicating in a YB-1 dependent manner, contains, as shown by analysis performed in connection with the present invention, both the promoter as well as the sequence for protein IX, as the 3′-UTR sequence contains the same. In tumor cells, however, the protein is only weakly expressed and results in a particle formation which is comparatively lower than the one of wildtype virus. The virus Xvir 03-03′UTR expresses the viral proteins E1B55k and E4orf6 by means of the heterologous CMV promoter (Clontech: Plasmid pShuttle) which is introduced into Xvir 03-03′UTR. Rather than the CMV promoter also those promoters as disclosed herein in connection with the expression of E1A may be used. The open reading frames of both genes are linked to each other by a so-called IRES sequence (internal ribosomal entry site) (Pelletier, J. and Sonenberg, N. Nature, 1988, 334, 320-325). This element (Novagen: pCITE) allows the expression of two proteins from one mRNA. A further option of the expression of two proteins from one RNA is the use of short peptides (2A) which are derived from food and mouth disease virus (Pablo de Felipe, Genetic Vaccines and Therapy, 2004, 2, 13). This element can, basically, be used in the various embodiments described herein as an alternative to the regulatory IRES sequence.

With regard to this regulatory background of the expression of protein IX in connection with YB-1 dependent replicating viruses and in particular adenoviruses which has been unknown prior to the filing of the present invention, the present inventor has realised that the expression of protein IX may, in principle, be ensured in YB-1 dependent replication and by viruses which replicate in a YB-1 dependent manner, by the following strategies:

1. By an independent promoter, particularly one by which protein E1A12S or protein E1B19K is controlled.

The independent promoter is preferably one which is different from the E1BIX promoter. Preferably, the independent promoter is selected from the group comprising tissue-specific, tumor-specific, YB-1-specific and viral promoters.

2. Control of the expression of protein IX by E1A12S. By the expression of the E1A12S protein an S phase induction of the infected cell occurs which results in the activation of protein IX by its natural promoter.

It is within the present invention that, in principle, promoters are used for the expression of the transporter which are different from the promoter which controls the expression of the transporter in the wildtype virus. In preferred embodiments this means that E1B55K is controlled by a promoter different from E1B, E4orf6 by a promoter different from the E4 promoter. In a further embodiment the promoter is one which is E1A independent, i.e. the activity of which is not influenced by E1A. Preferred promoters are thus tissue-specific promoters, tumor-specific promoters and viral promoters and in particular non-adenoviral vectors, preferably those described herein.

YB-1 dependent promoters which may be used in the present invention, include, but are not limited to: the adenoviral E2-late promoter, the MDR promoter [Stein et al, J. Biol. Chem, 2001, 276, 28562-28569;] as well as the DNA polymerase-alpha promoter [En-Nia et al, J. Biol. Chem., 2004, Epub ahead of print].

Non-adenoviral promoters which are suitable for the practising of the present invention may be selected from the group comprising cytomegalovirus promoter, RSV (Rous sarcoma virus) promoter, adenovirus-based promoter Va I and the non-viral YB-1 promoter (Makino Y. et al., Nucleic Acids Res. 1996, 15, 1873-1878). Further promoters which may be used in connection with any aspect of the invention disclosed herein, are the telomerase promoter, the alpha-fetoprotein (AFP) promoter, the caecinoembryonic antigen promoter (CEA) (Cao, G., Kuriyama, S., Gao, J., Mitoro, A., Cui, L., Nakatani, T., Zhang, X., Kikukawa, M., Pan, X., Fukui, H., Qi, Z. Int. J. Cancer, 78, 242-247, 1998), the L-plastin promoter (Chung, I., Schwartz, P E., Crystal, R C., Pizzorno, G, Leavitt, J., Deisseroth, A B. Cancer Gene Therapy, 6, 99-106, 1999), argenine vasopressin promoter (Coulson, J M, Staley, J., Woll, P J. British J. Cancer, 80, 1935-1944, 1999), E2f promoter (Tsukada et al., Cancer Res., 62, 3428-3477, 2002), uroplakin II promoter (Zhang et al., Cancer Res., 62, 3743-3750, 2002) and PSA promoter (Hallenbeck P L, Chang, Y N, Hay, C, Golightly, D., Stewart, D., Lin, J., Phipps, S., Chiang, Y L. Human Gene Therapy, 10, 1721-1733, 1999). Furthermore, the YB-1 dependent E2 late promoter of adenoviruses as described in German patent application DE 101 50 984.7 is a promoter which may be used in connection with the present invention.

From the telomerase promoter it is known that it is essential to human cells. The telomerase activity is controlled by the transcription control of the telomerase reverse transcriptase gene (hTERT) which is the catalytic subunit of the enzyme. The expression of the telomerase is active in 85% of human tumor cells. In contrast thereto it is inactive in most normal cells with the exception of germ cells and embryonal tissue (Braunstein, I. et al., Cancer Research, 61, 5529-5536, 2001; Majumdar, A. S. et al., Gene Therapy 8, 568-578, 2001). More detailed analyses of the hTERT promoter have shown that fragments of the promoter being 283 bp and 82 bp, respectively, away from the starting codon are sufficient for a specific expression in tumor cells (Braunstein I. et al.; Majumdar A S et al., supra). Therefore, this promoter and the specific fragments, respectively, are suitable for providing specific expression of a gene and in particular a transgene, preferably a transgene as disclosed herein, in tumor cells only.

Such a promoter is also to allow the expression of the modified oncogene, preferably the E1A oncogene protein, of the virus of the invention in tumor cells only. Also, in an embodiment the transgene, in particular one which is selected from the group comprising E4orf6, E1B55kD, ADP and YB-1 is expressed in an adenoviral vector under the control of one of these promoters. It is within the present invention that the reading frame of the transactivating oncogene protein, in particular of the E1A protein is in frame with one or several of the gene products of the adenoviral system. The reading frame of the transactivating E1A protein may, however, also be independent therefrom.

As used herein, the term transgene refers, in an embodiment, to all those genes which are either not contained in the virus, particularly the adenovirus of the wildtype and more preferably the adenovirus Ad5 wildtype, or in a different regulatory context, particularly as defined herein. The gene which is present in such a different regulatory context is herein also referred to as heterologous gene. It is within an embodiment of the present invention that one or several transgenes, as described herein, are coded and/or expressed by one or more of the helper genes.

The insights, methods, uses or nucleic acids, proteins, replication systems and the like, respectively, described herein are not necessarily limited to adenoviruses. In principle such systems exist also in other viruses which are encompassed herewith.

The use of the viruses according to the invention or the use in accordance with the present invention of the viruses described herein, may result in a replication comparable to the one of wildtype at an infection rate of 1 to 10 pfu/cell, compared to 10 to 100 pfu/cell according to the prior art.

The viruses according to the present invention allow a significantly increased particle formation compared to the YB-1 dependent viruses of the prior art. Preferably, particle formation is increased by the factor of 2 to 50, preferably the factor 10 to 50.

Finally, in an embodiment the adenoviruses used in accordance with the present invention are E1B deficient, in particular E1B 19K deficient. Deficient as generally used herein means a condition where E1B does not show the entirety of characteristics inherent to the wildtype and at least one of these characteristics is missing. The adenoviral BCL2 homologue E1B19k prevents E1A induced apoptosis by interaction with the pro-apoptotic proteins Bak and Bax. Because of this a maximum replication and/or particle formation is possible in infected cells (Ramya Sundararajan and Eileen White, Journal of Virology 2001, 75, 7506-7516). The lack of E1B 19k results in a better release of the viruses as, if present, it minimises the function of the adenoviral death protein. By such a deletion the cytopathic effect induced by the virus is increased (Ta-Chiang Liu et al., Molecular Therapy, 2004) and thus results in an enhanced lysis of the infected tumor cells. Additionally, the lack of E1B19K results in TNF-alpha not having an impact on the replication of such recombinant adenoviruses in tumor cells, whereas in normal cells the treatment with TNF-alpha results in decreased replication and release of infectious viruses. Thus selectivity and specificity is increased (Ta-Chiang Liu et al., Molecular Therapy 2004, 9, 786-803).

It is within the skills of the persons of the art to delete and mutate, respectively, adenoviral nucleic acid sequences which are non-essential for the invention. Such deletions may, for example, be related to nucleic acid coding for a part of the E3 and E4 as also described herein. In case E4 is deleted it is particularly preferred if such deletion does not extend to the protein E4orf6, in other words the adenovirus to be used in accordance with the present invention codes E4orf6. In preferred embodiments such adenoviral nucleic acids may still be packed into the viral capsid and thus infectious particle be formed. This applies equally to the use of the nucleic acids in accordance with the present invention. Generally it has also still to be noted that the adenoviral systems may be deficient with regard to single or several expression products. In connection therewith, it is to be noted that this may be based on the complete deletion or mutation of the nucleic acid coding for the expression product or on a deletion or mutation of such nucleic acid such that essentially no expression product is formed any longer or on the lack of regulatory and expression-controlling, respectively, elements such as promoters or transcription factors or on an activity of the same which is different from wildtype, be it at the level of the nucleic acids (lack of a promoter; cis acting element) or at the level of the translation and transcription system, respectively (trans-acting elements). Particularly the latter aspect may strongly depend on the respectively cellular background.

The YB-1 dependent replication of the viruses according to the invention occurs with regard to the replication of the adenoviruses of the wildtype as described in the following.

The replication of adenoviruses is a very complex process and is usually based on the human transcription factor E2F. During viral infection at first the “early genes” E1, E2, E3 and E4 are expressed. The group of the “late genes” is responsible for the synthesis of the structural proteins of the virus. The E1 region consisting of two transcriptional units E1A and E1B which code for different E1A and E1B proteins, plays a critical role for the activation of both the early and the late genes, as they induce the transcription of the E2, E3 and E4 genes (Nevins, J. R., Cell 26, 213-220, 1981). Additionally, the E1A proteins may initiate DNA synthesis in resting cells and thus trigger their entry into the S phase (c. f. Boulanger and Blair, 1991). Additionally, they interact with the tumor suppressors of the Rb class (Whyte, P. et al., Nature 334, 124-127, 1988). In doing so, the cellular transcription factor E2F is released. The E2F factors may subsequently bind to corresponding promoter regions of both cellular and viral genes (in particular to the adenoviral E2 early promoter) and initiate transcription and thus replication (Nevins, J. R., Science 258, 424-429, 1992).

The gene products of the E2 region are especially needed for the initiation and completion of the replication as they code for three essential proteins. The transcription of the E2 proteins is controlled by two promoters, the “E2 early E2F dependent” promoter, which is also referred to herein as E2-early promoter or early E2 promoter, and the “E2-late” promoter (Swaminathan and Thimmapaya, The Molecular Repertoire of Adenoviruses III: Current Topics in Microbiology and Immunology, vol. 199, 177-194, Springer Verlag 1995). Additionally, the products of the E4 region together with the E1A and E1B-55kDa protein play a crucial role for the activity of E2F and the stability of p53. For example, the E2 promoter is even more transactivated by direct interaction of the E4orf6/7 protein encoded by the E4 region, with the heterodimer consisting of E2F and DP1 (Swaminathan and Thimmapaya, J B C 258, 736-746, 1996). Furthermore, the complex consisting of E1B-55kDa and E4orf6 is inactivated by p53 (Steegenga, W. T. et al., Oncogene 16, 349-357, 1998) in order to complete a successful lytic infectious cycle. Additionally, E1B-55kDa has a further important function insofar as it promotes, when interacting with E4orf6 protein, the export of viral RNA from the nucleus, whereas cellular RNAs are retained in the nucleus (Bridge and Ketner, Virology 174, 345-353, 1990). A further important observation is that the protein complex consisting of E1B-55kDa/E4orf6 is localised in the so-called “viral inclusion bodies”. It is assumed that these structures are the sites of replication and transcription (Ornelles and Shenk, J. Virology 65, 424-429, 1991).

The E3 region is another important region for the replication and in particular for the release of adenoviruses. The E3 region more precisely contains the genetic information for a variety of comparatively small proteins which are not essential for the infectious cycle of adenovirus in vitro, i.e. in cell culture. However, they play a crucial role in the survival of the virus during an acute and/or latent infection in vivo as they have, among others, immunoregulatory and apoptotic function(s) (Marshall S. Horwitz, Virologie, 279, 1-8, 2001; Russell, supra). It could be shown that a protein having a size of about 11.6 kDa induces cell death. This protein was, due to its function, named ADP—for the english term adenovirus death protein—(Tollefson, J. Virology, 70, 2296-2306, 1996). The protein is predominantly formed in the late phase of the infectious cycle. Furthermore, the overexpression of the protein results in a better lysis of the infected cells (Doronin et al., J. Virology, 74, 6147-6155, 2000). In accordance therewith, the genes and proteins, respectively, are still contained in the virus in accordance with the present invention.

The use of the adenoviruses according to the present invention as medicaments and in particular in connection with systemic administration can be improved by a suitable targeting of the adenoviruses. The infection of tumor cells by adenovirus depends, among others, to a certain extent on the presence of the coxsackievirus-adenovirus receptor CAR and particular integrins. If these are strongly expressed in cells, in particular tumor cells, an infection is already possible at very low titers (pfu/cell). Different strategies have so far been followed in order to achieve a so called re-targeting of the recombinant adenoviruses by, for example, insertion of heterologous sequences into the fiber knob region and the C-terminus of protein IX, use of bi-specific antibodies, coating of the adenoviruses with polymers, introduction of ligands in the Ad fibre, substitution of the serotype 5 knob and serotype 5 fiber shaft and knob, respectively, by the serotype 3 knob and Ad35 fiber shaft and knob and modification of the penton base (Nicklin S. A. et al., Molecular Therapy 2001, 4, 534-542; Magnusson, M. K. et. al., J. of Virology 2001, 75, 7280-7289; Barnett B. G. et al., Biochimica et Biophysica Acta 2002, 1575, 1-14; Dimitrev I P et al., Journal of Virology, 2002, 76, 6893-6899; Mizuguchi und Hayakawa, Human Gene Therapy, 2004, 15, 1034-1044). Realizing such further designs and characteristics, respectively, in connection with the adenoviruses according to the present invention and the adenoviruses to be used in accordance with the invention, in their various aspects of the present invention, is within the present invention.

The various transgenes, including E1B55kD, E4orf6, ADP and the like, in particular if they are viral genes, may in principle be cloned from any respective virus, preferably adenovirus and more preferably adenovirus Ad5. A variety of plasmids are additionally described in the prior art which contain the respective genes and from which these may accordingly be taken and introduced into both the adenoviruses according to the present invention as well as the viruses to be used in accordance with the present invention. An example for a plasmid expressing E1B55kD is, for example, described by Dobbelstein, M. et al., EMBO Journal, 16, 4276-4284, 1997. The coding region of the E1B55K gene can, for example, can be excised together with the 3′ non-coding region (this 3′UTR region lies preferably at about base position 3507-4107 of the adenovirus wildtype genome) of this gene by means of Bam HI from the plasmid pDCRE1B. The respective fragment comprising the E1B55kD gene as well as the 3′ non-coding region corresponds to nucleotides 2019 to 4107 of the adenovirus type 5. It is, however, also within the present invention that the E1B55kD gene is excised from the plasmid by means of the restriction enzymes Bam HI and BfrI and XbaI, respectively, and subsequently cloned into the adenovirus. It is also within the present invention that also analogues thereof and in particular analogues of the 3′ UTR region may be used within the present invention. An analogue of the 3′ UTR region is any sequence which has the same effect as the 3′ UTR region, particularly the same effect with regard to the expression of a gene, preferably the E1B55kD gene. Such analogues can be determined by routine experiments performed by the persons skilled in the art, e.g. by extending or shortening the 3′ UTR region by one or several nucleotides and subsequently testing whether the thus obtained analogue still has the same effect as the 3′ UTR region as described previously. In an embodiment the term 3′ UTR region thus comprises also each and any analogue thereof.

Those viruses where therapeutic genes or transgenes are cloned preferably under the control of a specific promoter, in particular a tumor-specific or tissue-specific promoter, are further developments of the viruses according to the present invention. It is also within such viruses that also the E4 region is functionally inactive and is preferably deleted. The transgenes described herein can also be cloned into the E4 region, whereby this may be performed alternatively or additionally to the cloning of the transgenes into the E3 region and the E3 region may remain partially or completely intact, respectively. Transgenes as used herein may be therapeutic genes or viral genes, preferably adenoviral genes, which are preferably not present in the genome of wildtype adenoviruses and which are not present, respectively, at the site of the genome at which they are located in the particular virus now.

Therapeutic genes can be prodrug genes, genes for cytokines, apoptosis inducing genes, tumor suppressor genes, genes for metalloproteinase inhibitors and/or angiogenesis inhibitors, and tyrosine kinase inhibitors. Further siRNA, aptamers, antisense molecules and ribozymes may be expressed which are preferably directed against cancer-relevant target molecules. Preferably the individual or the several target molecules are selected from the group comprising the resistance-relevant factors, anti-apoptosis factors, oncogenes, angiogenesis factors, DNA synthesis enzymes, DNA repair enzymes, growth factors and their receptors, transcription factors, metalloproteinases, particularly matrix metalloproteinases, and plasminogen activator of the urokinase type. Preferred embodiments thereof are already disclosed herein.

In an embodiment the resistance-relevant factors are preferably selected from the group comprising P-glycoprotein, MRP and GST and also comprise the nucleic acids coding therefor.

Possible prodrug genes as may be used in preferred embodiments, are, for example, cytosine deaminase, thymidine kinase, carboxypeptidase, uracil phosphoribosyl transferase; or purine nucleoside phosphorylase (PNP); Kim et al, Trends in Molecular Medicine, volume 8, no. 4 (suppl), 2002; Wybranietz W. A. et al., Gene Therapy, 8, 1654-1664, 2001; Niculescu-Duvaz et al., Curr. Opin. Mol. Therapy, 1, 480.486, 1999; Koyama et al., Cancer Gene Therapy, 7, 1015-1022, 2000; Rogers et al., Human Gene Therapy, 7, 2235-2245, 1996; Lockett et al., Clinical Cancer Res., 3, 2075-2080, 1997; Vijayakrishna et al., J. Pharmacol. And Exp. Therapeutics, 304, 1280-1284, 2003.

Possible cytokines as may be used in preferred embodiments, are, for example, GM-CSF, TNF-alpha, Il-12, Il-2, Il-6, CSF or interferon-gamma; Gene Therapy, Advances in Pharmacology, volume 40, editor: J. Thomas August, Academic Press; Zhang and Degroot, Endocrinology, 144, 1393-1398, 2003; Descamps et al., J. Mol. Med., 74, 183-189, 1996; Majumdar et al., Cancer Gene Therapy, 7, 1086-1099, 2000.

In an embodiment the anti-apoptosis factors are selected from the group comprising BCL2 and comprise also the nucleic acids coding therefor. In an embodiment the oncogenes are selected from the group comprising Ras, particularly mutated Ras, Rb and Myc, and comprises also the nucleic acids coding therefor. In an embodiment the angiogenesis factors are selected from the group comprising VEGF and HMG proteins, and also comprise the nucleic acids coding therefor. In an embodiment the DNA synthesis enzymes are selected from the group comprising telomerase, and also comprise the nucleic acids coding therefor. In an embodiment the DNA repair enzymes are selected from the group comprising Ku-80, and also comprise the nucleic acids coding therefor. In an embodiment the growth factors are selected from the group comprising PDGF, EGF and M-CSF, and also comprise the nucleic acids coding therefor. In a further embodiment the receptors are in particular those of growth factors, whereby preferably the growth factors are selected from the group comprising PDGF, EGF and M-CSF, and also comprise the nucleic acids coding therefor. In an embodiment the transcription factor is selected from the group comprising YB-1, and also comprises the nucleic acid coding therefor. In an embodiment the metalloproteinases are in particular matrix metalloproteinases. In a preferred embodiment the matrix metalloproteinases are selected from the group comprising MMP-1 and MMP-2, and also comprise the nucleic acids coding therefor. In an embodiment the plasminogen activators of the urokinase type are selected from the group comprising uPa-R, and also comprise the nucleic acids coding therefor.

Possible apoptosis-inducing genes as may be used in preferred embodiments, are, for example, Decorin:Tralhao et al., FASEB J, 17, 464-466, 2003; retinoblastoma 94: Zhang et al., Cancer Res., 63, 760-765, 2003; Bax and Bad: Zhang et al., Hum. Gene Ther., 20, 2051-2064, 2002; apoptin: Noteborn and Pietersen, Adv. Exp. Med. Biol., 465, 153-161, 2000; ADP: Toth et al., Cancer Gene Therapy, 10, 193-200, 2003; bcl-xs: Sumantran et al., Cancer Res, 55, 2507-2512, 1995; E4orf4: Braithwaite and Russell, Apoptosis, 6, 359-370, 2001; FasL, Apo-1 and Trail: Boehringer Manheim, Guide to Apoptotic Pathways, Arai et al., PNAC, 94, 13862-13867, 1997; Bims: Yamaguchi et al., Gene Therapy, 10, 375-385, 2003; GNR163: Oncology News, 17 Jun., 2000.

Possible tumor suppressor genes as may be used in preferred embodiments, are, for example, E1A, p53, p16, p21, p27 or MDA-7: Opalka et al., Cell Tissues Organs, 172, 126-132, 2002, Ji et al., Cancer Res., 59, 3333-3339, 1999, Su et al., Oncogene, 22, 1164-1180, 2003.

Possible angiogenesis inhibitors as may be used in preferred embodiments, are, for example, endostatin or angiostatin: Hajitou et al., FASEB J., 16, 1802-1804, 2002, and antibodies against VEGF: Ferrara, N., Semin Oncol 2002 December; 29 (6 suppl 16): 10-4.

Possible metalloproteinase inhibitors as may be used in preferred embodiments, are, for example, Timp-3 (Ahonen et al., Mol Therapy, 5, 705-715, 2002); PAI-1 (Soff et al., J. Clin. Invest., 96, 2593-2600, 1995); Timp-1 (Brandt K. Curr. Gene Therapy, 2, 255-271, 2002).

Further transgenes in the sense of the present invention which may be expressed by the viruses according to the present invention are also tyrosine kinase inhibitors. Exemplary tyrosine kinases are EGFR (epidermal growth factor receptor) [Onkologie, Entstehung und Progression maligner Tumoren; author: Christoph Wagner, Georg Thieme Verlag, Stuttgart, 1999]. A preferred tyrosine kinase inhibitor is herceptin [Zhang H et al., Cancer Biol Ther. 2003, July-August; 2 (4 suppl 1): S122-6].

SiRNA (short interfering RNA), as may be used within the present invention, consists of two, preferably separate RNA strands which hybridise to each other due to base complementarity which means that they are present essentially base paired and preferably have a length of up to 50 nucleotides, preferably between 18 and 30 nucleotides, more preferably less than 25 nucleotides and most preferably 21, 22 or 23 nucleotides, whereby these figures refer to the single strand of the siRNA, particularly to the length of the stretch of the single strand which hybridises to or is base paired with a, more precisely the second single strand. siRNA specifically induces or mediates the degradation of mRNA. The specificity required theretofore is mediated by the sequence of the siRNA and thus its binding site. The target sequence to be degraded is essentially complementary to the first or to the second of the siRNA forming strands. Although the precise mode of action is not yet clear, it is assumed that siRNA is a biological strategy for cells in order to inhibit distinct alleles during development and to protect themselves against viruses. siRNA mediated RNA interference is used as a method for the specific suppression or complete elimination of the expression of a protein by introducing a gene specific double-stranded RNA. For higher organisms a siRNA comprising 19 to 23 nucleotides is insofar particularly suitable as it does not result in the activation of a non-specific defense reaction such as an interleukin response. The direct transfection of double-stranded RNA of 21 nucleotides having symmetrical 2-nt 3′ overhangs was suitable to mediate RNA interference in mammalian cells and is highly efficient compared to other technologies such as ribozymes and antisense molecules (Elbashir, S. Harborth J. Lendeckel W. Yalvcin, A. Weber K, Tuschl T: Duplexes of 21-nucleotide RNAs mediate RNA interference in cultured mammalian cells. Nature 2001, 411: 494-498). As little as a few siRNA molecules are sufficient so as to suppress expression of the target gene. In order to avoid the limitations of exogenously added siRNA which particularly reside in the transient nature of the interference phenomenon and specific delivery (delivery) of the siRNA molecules, vectors are used in the prior art which allow for an endogenous siRNA expression. For such purpose, for example, oligonucleotides having a length of 64 nucleotides are introduced into the vector which comprise the 19 nucleotide long target sequence both in the sense and in the antisense orientation, separated by, for example, a 9 nucleotide spacer sequence. The resulting transcript folds into a hairpin structure with a stem structure (stem) of, for example, 19 base pairs. The loop is rapidly degraded in the cell so that a functional siRNA molecule is generated (Brummelkamp et al., Science, 296, 550-553, 2002).

In a further aspect the present invention relates to a medicament which comprises at least a virus according to the present invention. In a further aspect the present invention is related to the use of the viruses of the invention for the manufacture of a medicament. In connection therewith the medicament is for the treatment of tumors and cancer diseases. The tumors and cancer diseases are preferably those described herein. Preferably the tumor and cancer diseases are those which are and have, respectively, a resistance. Preferably the resistance is one as described herein, particularly preferably a multiple resistance, a multiple resistance against cytostatics and/or radiation. In a further aspect the medicament is for restoring of the sensitivity of cells towards cytostatics and/or radiation, whereby the cells are preferably tumor cells which show resistance against cytostatics and/or radiation. The restoration of the sensitivity is a process which is referred to in English as “restoration of drug sensitivity”.

In a still further embodiment the medicament further comprises at least one pharmaceutically active compound.

In a preferred embodiment the pharmaceutically active compound is selected from the group comprising cytokines, metalloproteinase inhibitors, angiogenesis inhibitors, cytostatics such as Irinotecan and CPT-11 against colorectal carcinoma and daunorubicin against leukemia, cell cycle inhibitors such as CYC202 which inhibits CDK2/CyclinE kinase activity and can be used against colorectal tumors (McClue S J, Int. J. Cancer 2002, 102, 463-468) and BAY 43-9006 which inhibits Raf-1 and is, for example, effective against mamma carcinoma (Wilhelm S M et al., Cancer Res. 2004, 64, 7099-7109), proteosome inhibitors such as PS-341 which inhibits the 26S proteasome activity and is used against squamous cell carcinoma (Fribley A et al., Mol Cell Biol 2004 November; 24(22): 9695-704), recombinant antibodies directed against, for example, the EGF receptor (Herceptin for breast carcinoma and prostate tumor; H. G. van der Poel, European Urology 2004, 1-17; Erbitux against head and neck tumors; Bauman M et al., Radiother. Oncol., 2004, 72, 257-266), and inhibitors of the signal transduction cascade such as STI 571 which represses, among others, c-kit and can be used against gastrointestinal tumors (H. G. van der Poel, European Urology 2004, 45, 1-17), ABT-627 which is an endothelin inhibitor and which may be used, among others, against prostate tumors (H. G. van der Poel, European Urology 2004, 45, 1-17), SU5416 which inhibits phosphorylation of the VEGF tyrosine kinase receptor and which may be used, among others, against glioblastoma and prostate cancer (Bischof M et al Int. J. Radiat. Oncol. Biol. Phys. 2004; 60 (4): 1220-32), ZD1839 which inhibits EGFR tyrosine activity and may be used, among others, against prostate tumors (H. G. van der Poel, European Urology 2004, 45, 1-17); rapamycine derivatives such as CCI-779 and RAD001 which inhibit mTOR and can be used against prostate tumors. It is within the present invention that the various adenoviruses described herein and the adenoviruses to be used in accordance with the present invention, respectively, can, in principle, be used with each and any of the aforementioned compounds for each and any of the indication described in connection therewith. In a particularly preferred embodiment the indication is the one which is described for any of the previously mentioned pharmaceutically active compounds.

The present inventor has further surprisingly found that the efficacy of the viruses described herein and in particular the viruses used in accordance with the present invention can be increased by using it in combination with at least two compounds whereby each of the at least two compounds is individually and independently selected from the group comprising cytostatics. The compounds are in a preferred embodiment pharmaceutically active compounds.

As used herein in a preferred embodiment, cytostatics are in particular chemical or biological compounds which, during or after the administration to a cell or an organism containing a or such cell, cause that the cell no longer grows and/or no longer divides or cell division and/or cell growth is slowed down. Cytostatics also comprise compounds which turn into a cytostatic in the aforedescribed sense only in the cell or in an organism containing such cell. Insofar, the term cytostatics also comprises pre-cytostatics.

Cytostatics are grouped according to their mode of action. The following groups are distinguished which, in principle, can all be used within the present invention:
Alkylating agents, i.e. chemical compounds which cause their cytotoxic effect by alkylating phosphate, amino, sulphydryl, carboxy and hydroxy groups of the nucleic acid as well as proteins. Such compounds are often cancerogenic themselves. Typical examples of this group of cytostatics are cis-platin and platin derivatives, cyclophosphamide, dacarbazine, mitomycin, procarbazine.
Antimetabolites, i.e. compounds which, due to their structural similarity or ability for binding block a metabolic process or affect the same. Within the group of antimetabolites it is distinguished between structurally similar antimetabolites, structure changing antimetabolites and the indirectly acting antimetabolites. The structurally similar antimetabolites compete due to chemical similarity with the metabolite without exerting the function thereof. Structure changing antimetabolites bind to the metabolites which impedes its function or resorption or chemically modifies the metabolite. Indirectly acting antimetabolites interfere with the function of the metabolite, for example by the binding of ions. Typical examples of this group are folic acid antagonists such as methotrexate, pyrimidine analogues such as fluorouracil, purine analogues such as azathioprine and mercaptopurine.
Mitosis inhibitors, i.e. compounds which inhibit cell division. Within the group of mitosis inhibitors it is distinguished between cell division toxins, spindle toxins and chromosome toxins. Typical examples of this group are taxanes and vinca alkaloids. The taxanes in turn can be divided into the two major groups of taxoles and taxoters, whereby a particularly preferred taxole is paclitaxel, and a particularly preferred taxoter is docetaxel.
Antibiotics having an inhibitory effect on the DNA-dependent RNA polymerase. Typical examples are the anthracyclines, such as, e.g., bleomycin, daunorubicin, doxorubicin and mitomycin.
Topoisomerase inhibitors, in particular topoisomerase I inhibitors. Topoisomerase inhibitors are chemical compounds which determine the tertiary structure of the DNA by catalysing the change of the DNA twist number in a three stage process. Essentially, two forms of topoisomerases are distinguished. Topoisomerases of type I cleave only a DNA strand and are ATP-independent, whereas topoisomerase of type II cleave both strands of a DNA, whereby they are ATP-dependent. Typical examples for topoisomerase I inhibitors are irinotecan and topotecan, and for topoisomersae II inhibitors etoposid and daunorubicin.

Within the present invention at least one and preferably two agents are selected from the aforementioned group. It is, however, also within the invention that in particular also three, four or five different agents are selected. The following comments are made for the embodiment of the present invention where only one and preferably two agents are used together with the virus. These considerations are basically also applicable to the embodiments where more than two agents are used.

Preferably the agents differ from each other such that they address different target molecules or are described in literature as targeting different molecules. It is within the present invention that the agent also comprises two or more different compounds which bind to the same target molecule. It is also within the present invention that one agent binds to a first site of the target molecule, whereas the second agent binds to a second site of the target molecule.

It is also within the present invention that at least two of the agents are active using different modes of action. Active means in a preferred embodiment that the cell growth and/or cell division inhibiting or retarding effect of the chemical compound is mediated through a different mode of action. In a particularly preferred embodiment the term active means that the replication efficiency of a virus, in particular the virus according to the present invention, of the viruses described herein and of the viruses to be used in accordance with the present invention, is increased compared to a scenario where one and/or both of the agents are not used. As a measure for the efficiency of viral replication preferably the number of viruses required for cell lysis is used, more preferably expressed as pfu/cell.

In a particularly preferred embodiment at least one of the at least two agents is one which increases the infectability of the cell in which the replication of the virus is to occur, preferably is to occur in a selective manner, preferably with the virus described herein and/or the virus to be used in accordance with the present invention. This can, e.g., be performed by increasing the uptake of the virus by the cell. The uptake of the virus, in particular of adenovirus, is, for example, mediated by the coxsackievirus-adenovirus receptor (CAR) (Mizuguchi und Hayakawa, GENE 285, 69-77, 2002). An increased expression of CAR is, for example, caused by trichostatin A (Vigushin et al., Clinical Cancer Research, 7, 971-976, 2001).

In a further embodiment one of the at least two agents is one which increases the availability of a component within the cell, whereby the component is one which increases the replication of the virus, preferably the virus described herein and/or the virus to be used in accordance with the present invention.

In a further embodiment one of the at least two agents is one which mediates the transport of YB-1 into the nucleus. Such an agent can be selected from the group comprising topoisomerase inhibitors, alkylating agents, antimetabolites and mitosis inhibitors. Preferred topoisomerase inhibitors are camptothecin, irinotecan, etoposide and their respective analogues. Preferred mitosis inhibitors are daunorubicin, doxorubicin, paclitaxel and docetaxel. Preferred alkylating agents are cis-platin and their analogues. Preferred antimetabolites are fluorouracil and methotrexat.

In a particularly preferred embodiment one of the at least two agents is one which increases the infectability of the cell, in particular the expression of CAR, and the second of the at least two agents is one which increases the transport of YB-1 into the nucleus, whereby preferably as chemical compound a compound is used which exhibits the respective required characteristic as preferably described above. An example for the class of compounds increasing the expression of CAR are histone deacetylase inhibitors and an example for a class of compounds increasing the transport of YB-1 into the nucleus are topoisomerase inhibitors.

In a further embodiment one of the at least two agents is a histone deacylase inhibitor and the other one of the at least two agents is a topoisomerase inhibitor.

In a further embodiment one of the at least two agents is a histone deacylase inhibitor. A preferred histone deacylase inhibitor is one which is selected from the group comprising trichostatin A, FR901228, MS-27-275, NVP-LAQ824 and PXD101. Trichostatin A is, for example, described in Vigushin et al., Clinical Cancer Research, 7, 971-976, 2001; FR901228 is, for example, described in Kitazono et al., Cancer Res., 61, 6328-6330, 2001; MS-27-275 is described in Jaboin et al., Cancer Res., 62, 6108-6115, 2002; PXD101 is described in Plumb et al., Mol. Cancer Ther., 8, 721-728, 2003; NVP-LAQ824 is described in Atadja et al., Cancer Res., 64, 689-695, 2004.

In an embodiment at least one agent is selected from the group comprising trichostatin A (against glioblastoma, Kim J H et al., Int. J. Radiation Oncology Biol. Phys. 2004, 59, 1174-1180), FR 901228 (against pancreas tumors, Sato N et al., Int. J. Oncol. 2004, 24, 679-685; MS-27-275 (against prostate tumors; Camphausen K et al., Clinical Canver Research 2004, 10, 6066-6071), NVP-LAQ824 (against leukemiae; Nimmanapalli R et al., Cancer Res. 2003, 63, 5126-5135); PXD101 (against ovary tumors, Plumb J A et al, Mol. Cancer Ther. 2003, 2, 721-728), scriptaid (against breast carcinoma, Keen J C et al., Breast Cancer Res. Treat. 2003, 81, 177-186), apicidin (against melanoma, Kim S H et al., Biochem. Biophys. Res. Commun. 2004, 315, 964-970) and CI-994 (against various tumors, Nemunaitis J J et al., Cancer J. 2003, 9, 58-66). The mode of action of histone deacetylase inhibitors is described, among others, in Lindemann R K et al., Cell Cycle 2004, 3, 77-86. It is within the present invention that the various adenoviruses described herein and the adenoviruses to be used in accordance with the present invention, may be used with the aforementioned compounds, in principle, for each and any of the indications described herein in connection therewith. In a particularly preferred embodiment the indication is one as has been described for each and any of the aforementioned pharmaceutically active compounds.

In a still further embodiment one of the at least two agents is a topoisomerase inhibitor, preferably a topoisomerase I inhibitor. A preferred topoisomerse inhibitor is one which is selected from the group comprising camptothecin, irinotecan, topotecan, SN-38, 9-aminocamptothecin, 9-nitrocamptothecin, DX-895If and daunorubicin. Irinotecan and SN-38 are, for example, described in Gilbert et al., Clinical Cancer Res., 9, 2940-2949, 2003; DX-895IF is described in van Hattum et al., British Journal of Cancer, 87, 665-672, 2002; camptothecin is described in Avemann et al., Mol. Cell. Biol., 8, 3026-3034, 1988; 9-aminocamptothecin, 9-nitrocamptothecin are described in Rajendra et al., Cancer Res., 63, 3228-3233, 2003; daunorubicin is described in M. Binaschi et al., Mol. Pharmacol., 51, 1053-1059.

In a preferred embodiment the topoisomerase inhibitor is selected from the group comprising camptothecin, irinotecan, topotecan, DX-895If, SN-38, 9-aminocamptothecin, 9-nitrocamptothecin, etoposid and daunorubicin. These may be used against various tumors, for example, colorectal tumors, pancreas tumors, ovary carcinomas and prostate carcinomas. The fields of application are, among others, described by Recchia F et al., British J. Cancer 2004, 91, 1442-1446; Cantore M et al., Oncology 2004, 67, 93-97; Maurel J. et al., Gynecol. Oncol 2004, 95, 114-119; Amin A. et al., Urol. Oncol. 2004, 22, 398-403; Kindler H L et al., Invest. New Drugs 2004, 22, 323-327, Ahmad T. et al., Expert Opin. Pharmacother. 2004, 5, 2333-2340; Azzariti A. et al., Biochem Pharmacol. 2004, 68, 135-144; Le Q T et al., Clinical Cancer Res. 2004, 10, 5418-5424. It is within the present invention that the various adenoviruses described herein and the adenoviruses to be used in accordance with the present invention, respectively, may in principle be used with the aforementioned compounds for each and any of the indications described herein in connection therewith. In a particularly preferred embodiment the indication is such as described for each of the aforementioned pharmaceutically active compounds.

In a preferred embodiment of each and any aspect of the present invention the further pharmaceutically active compound is selected from the group comprising cytokines, metalloproteinase inhibitors, angiogenesis inhibitors, cytostatics such as irinotecan and CPT-11 against colorectal carcinoma and daunorubicin against leukemia, cell cycle inhibitors such as CYC202 which inhibits CDK2/CyclinE kinase activity and can be used against colorectal tumors (McClue S J, Int. J. Cancer 2002, 102, 463-468) and BAY 43-9006 which inhibits Raf-1 and is effective against mamma carcinoma (Wilhelm S M et al., Cancer Res. 2004, 64, 7099-7109), proteosome inhibitors such as PS-341 which inhibits the 26S proteasome activity and is used against brain tumors (Yin D. et al., Oncogene 2004), recombinant antibodies such as against the EGF receptor (Herceptin for breast carcinoma and prostate tumor; H. G. van der Poel, European Urology 2004, 1-17; Erbitux against head and neck tumors; Bauman M et al., Radiother. Oncol., 2004, 72, 257-266), and inhibitors of the signal transduction cascade such as STI 571 which represses, among others, c-kit and can be used against gastrointestinal tumors (H. G. van der Poel, European Urology 2004, 45, 1-17), ABT-627 an endothelin inhibitor which may be used, among others, against prostate tumors (H. G. van der Poel, European Urology 2004, 45, 1-17), SU5416 which inhibits phosphorylation of the VEGF tyrosine kinase receptor and which may be used against head/neck tumors (Cooney et al., Cancer Chemother. Pharmacol 2004), ZD1839 which inhibits EGFR tyrosine activity and may be used, among others, against prostate tumors (H. G. van der Poel, European Urology 2004, 45, 1-17); rapamycin derivatives such as CCI-779 and RAD001 which inhibit mTOR and can be used against prostate tumors (H. G. van der Poel, European Urology 2004, 45, 1-17). It is within the present invention that the various adenoviruses described herein and the adenoviruses to be used in accordance with the present invention, respectively, can, in principle, be used with each and any of the aforementioned compounds for each and any of the indications described in connection therewith. In a particularly preferred embodiment the indication is the one which is described for any of the previously mentioned pharmaceutically active compounds.

In an embodiment the medicament of the invention and/or the medicament prepared in accordance with the invention contains the virus separated from one or several of the at least one and preferably at least two agents which are combined with the virus in accordance with the present invention. The agents are preferably pharmaceutically active compounds. It is preferred that the virus is separated from any agent which is combined with the virus. Preferably the separation is a spatial separation. The spatial separation can be such that the virus is present in a different package than the agent(s). Preferably the package is a single dose unit, i.e. the virus and/or the agent(s) is/are packed as single dose unit. The single dose units may in turn be combined to form a package. However, it is also within the present invention that the single dose units of the virus are combined with one or several single dose units of one or several of the agents or packed therewith.

The kind of package depends on the way of administration as known to the one skilled in the art. Preferably the virus will be present in a lyophilized form or in a suitable liquid phase. Preferably, the agents will be present in solid form, e.g. as tablets or capsules, however, are not limited thereto. Alternatively, also the agents can be present in liquid form.

It is within the present invention that the virus is systemically or locally administered. It is also within the present invention that the agents combined with the virus are systemically or locally administered individually and independently from each other or together. Other modes of administration are known to the persons skilled in the art.

It is within the present invention that the virus and the agents combined with it, are administered in a chronologically separate manner or at the same time. In connection with a chronologically separate manner it is preferred that the agent is administered prior to the administration of the virus. How long the agent is administered prior to the virus depends on the kind of the agent used and is obvious for the person skilled in the art from the mode of action of the agent used. In case of administration of the virus in combination with at least two agents, the administration of the at least two agents can occur at the same or at different points in time. In connection with a chronologically different administration the points of time again result from the modes of action underlying the agents and can, based thereon, be determined by the persons skilled in the art.

The above considerations, given in connection with the medicaments according to the present invention which are also referred to herein as pharmaceutical compositions, are roughly also applicable to any composition, including compositions as used for the replication of viruses, preferably for the in vitro replication of viruses in accordance with the present invention. The above considerations are also applicable to the kit according to the present invention and the kit to be used in accordance with the present invention, respectively, which may apart from the viruses described herein and the viruses to be used in accordance with the invention, also comprise an agent or a combination of agents as described herein. Such kits comprise the virus and/or the one or the several agents in a form ready for use and preferably instructions for use. Furthermore, the above embodiments apply also to the nucleic acids as disclosed herein, and the nucleic acids used in accordance with the present invention, and the replication systems in accordance with the present invention and the nucleic acids coding therefor, and the replication systems used in accordance with the present invention and the nucleic acids coding therefor used in accordance with the present invention, and vice versa.

The medicament in connection with which or for the manufacture of which the adenoviruses disclosed herein are used in accordance with the present invention, is intended to be applied, usually, in a systemic manner, although it is also within the present invention to apply or deliver it locally. The application is intended to infect particularly those cells with adenoviruses and it is intended that adenoviral replication particularly occurs therein, which are involved, preferably in a causal manner, in the formation of a condition, typically a disease, for the diagnosis and/or prevention and/or treatment of which the medicament according to the present invention is used.

Such a medicament is preferably for the treatment of tumor diseases. Those tumor diseases are particularly preferred where either YB-1 is, due to the mechanism underlying the tumor disease, in particular due to the underlying pathological mechanism, already located in the nucleus, which have deregulated YB-1, or where the presence of YB-1 in the nucleus is caused by exogenous measures whereby such exogenous measures are suitable to transfer YB-1 into the cellular nucleus or to induce or to express it there. The term tumor or tumor disease shall comprise herein both malignant as well as benign tumors, and respective diseases. In an embodiment the medicament comprises at least one further pharmaceutically active compound. The nature and the amount of such further pharmaceutically active compound will depend on the kind of indication for which the medicament is used. In case the medicament is used for the treatment and/or prevention of tumor diseases, typically cytostatics such as, but not limited to, cis-platin and taxole, daunoblastin, daunorubicin, adriamycin (doxorubicin) and/or mitoxantrone or others of the cytostatics or groups of cytostatics described herein are used.

The medicament according to the invention can be present in various formulations, preferably in a liquid form. Furthermore, the medicament will contain adjuvants such as stabilisers, buffers, preservatives and the like which are known to the one skilled in the art of formulations.

The present inventor has surprisingly found that the viruses of the invention may be used with a high success rate in connection with those tumors where YB-1 is present in the nucleus independent of the cell cycle and those tumors which contain deregulated YB-1. Normally, YB-1 is present in the cytoplasm, in particular also in the perinuclear plasma. In the S phase of the cell cycle, YB-1 is in the nucleus of both normal as well as tumor cells. This, however, is not sufficient in order to provide a viral oncolysis using such modified adenoviruses. The comparatively little efficacy of such attenuated viruses as described in the prior art is ultimately based on their improper use. In other words, such adenoviral systems, could be used, particularly also with a higher efficacy, where the molecular biological prerequisites are given for viral oncolysis using these attenuated or modified viruses which are described herein. Such prerequisites are given in connection with the aforedescribed tumor diseases, i.e. those tumor diseases the cells of which show a nuclear localisation of YB-1 independent of the cell cycle, or have deregulated YB-1. This kind of nuclear localisation may be caused by the nature of the tumor itself, or by the agents according to the present invention as described herein, including the viruses described herein, or by other measures. The present invention thus defines a new group of tumors and tumor diseases and thus also of patients which may still effectively be treated using the viruses according to the present invention and in particular also using the attenuated or modified adenoviruses as already described in the prior art.

Without wishing to be bound in the following, it seems that “deregulated” YB-1 is an overexpressed or phosphorylated YB-1. This is based on the following facts. Akt which is a serine/threonine kinase, promotes the growth of tumor cells by phosphorylation of transcription factors and cell cycle proteins (Nicholson K M and Anderson N G, Cell. Signal., 14, 381-395, 2002). Additionally, one has found that activated Akt (phosphorylated Akt) correlates in a positive manner with YB-1 and that Akt is binding to YB-1 and phosphorylates the same at position Ser 102 of the cold-shock domain (Sutherland B W et al., Oncogene, 24, 4282-4292, 2005). This is indicating that there are signal transduction pathways which change the subcellular localisation of YB-1 and as such immediately its function. Additionally, this phosphorylation increases the production of proteins such as MDR and MRP, which are involved in stress response, cell proliferation and oncogenic transformation (Evdokimova V et al., Molecular and Cellular Biology, 26, 277-292, 2006). The phosphorylation of YB-1 by Akt, however, weakens its Cap binding capacity, whereby a translational activation of silent mRNA species is made easier (Evdokimova V et al., Molecular and Cellular Biology, 26, 277-292, 2006). As Akt is not active in normal cells, YB-1 is not present in a phosphorylated form, whereas YB-1 is deregulated, i.e. phosphorylated and/or overexpressed in such cells.

A further group of tumors and tumor diseases and thus of patients which may be treated using the viruses according to the invention and thus the medicament containing the same, are those whereupon applying or realising certain conditions it is ensured that YB-1 migrates into the nucleus or is induced or transported thereto, including by using the virus according to the invention or the viruses used in accordance with the present invention. This use of the viruses in connection with these tumors and patient groups, respectively, is based on the finding that the induction of the viral replication is based on nuclear localisation of YB-1 with subsequent binding of YB-1 to the E2-late promoter. This is also true for those cells which are YB-1 nucleus-positive and/or cells where YB-1 is present in a deregulated manner in the sense of the present invention. Insofar the adenoviruses according to the present invention can be used in accordance with the present invention for the treatment of diseases and groups of patients, respectively, which have cells having these characteristics, particularly if these cells are involved in the development of the respective disease to be treated. A further group of patients which can be treated by using such viruses, in particular adenoviruses, are thus those which are YB-1 nucleus-positive as a result of the subsequently described treatments and/or patients which have undergone one of the measures described herein, preferably in the sense of a treatment, or have experienced the administration of the viruses according to the present invention or experience them together with the administration of the virus according to the present invention. It is within the present invention that YB-1 nucleus-positive patients are patients which have YB-1 in the nucleus independent of the cell cycle, in particular in a number of tumour forming cells. These measures comprise the administration of such cytostatics as they are generally described herein and/or as they are used in connection with tumour therapy. Furthermore this group of measures comprises irradiation, in particular irradiation as used in connection with tumour therapy. Irradiation means in particular irradiation with energy-rich radiation, preferably radioactive radiation, preferably as used in connection with tumour therapy. A further measure is hyperthermia and the application of hyperthermia, preferably hyperthermia as used in connection with tumour therapy. In a particularly preferred embodiment hyperthermia is applied locally. Finally, a further measure is hormone treatment, in particular hormone treatment as used in connection with tumour treatment. In connection with such hormone treatment anti-estrogens and/or anti-androgens are used. In connection therewith, anti-estrogens such as Tamoxifen, are particularly used in the therapy of breast cancer, and anti-androgens as, for example, Flutamide and cyproterone acetate, are used in the therapy of prostate cancer.

It is within the present invention that some of the tumor forming cells which either inherently contain YB-1 in the nucleus or do so or after induction and active introduction into the nucleus or which comprise deregulated YB-1 in the meaning of the present disclosure. Preferably about 5% or any percentage higher than that, i.e. 6%, 7%, 8% etc., of the tumor forming cells are such YB-1 nucleus-positive cells or cells in which deregulated YB-1 is present. Nuclear localisation of YB-1 may be induced by outside stress and locally applied stress, respectively. This induction may occur through irradiation, particularly UV-irradiation, application of cytostatics as, among others, also disclosed herein, and hyperthermia. In connection with hyperthermia it is important that it may be realized in a very specific manner nowadays, particularly in a specific local manner, and that thus also a specific nuclear transport of YB-1 into the nucleus may be caused and, because of this, the prerequisites for replication of the adenovirus and thus of cell and tumor lysis are given, which preferably is locally limited (Stein U, Jurchott K, Walther W, Bergmann, S, Schlag P M, Royer H D. J Biol Chem. 2001, 276(30):28562-9; Hu Z, Jin S, Scotto K W. J Biol Chem. 2000 Jan. 28; 275(4):2979-85; Ohga T, Uchiumi T, Makino Y, Koike K, Wada M, Kuwano M, Kohno K. J Biol Chem. 1998, 273(11):5997-6000).

With regard to the characteristics of the cells for the lysis of which the adenoviruses described herein are used in accordance with the present invention, it is envisaged that these show, in an embodiment, a resistance, preferably a multiple resistance or poly-resistance. Resistance as used herein preferably refers to a resistance to cytostatics and in particular to the cytostatics described herein, and/or radiation. This multiple resistance preferably goes along with the expression, preferably an overexpression of the membrane-bound transport protein P-glycoprotein which is a marker for the determination of respective cells and thus also of respective tumors and the corresponding patient groups, respectively. The term resistance as used herein comprises both the resistance which is also referred to as classical resistance and which is mediated by the P-glycoprotein, as well as the resistance which is also referred to as atypical resistance and which is mediated by MRP or other, non-P-glycoprotein mediated resistances. Further resistances to which it is referred to herein and which are characteristic for the tumors and patients, respectively, to be treated in accordance with the present invention, are those which are mediated by the following genes, however, are not limited thereto: MDR, MRP, topoisomerase, BCL2, glutathione-S-transferase (GST), protein kinase C (PKC). As the effect of cytostatics is, among others, based on the induction of apoptosis, the expression of apoptosis relevant genes plays an important role in the formation of resistance so that also the following factors are relevant insofar, namely Fas, the BCL2-family, HSP70 and EGFR [Kim et al., Cancer Chemther. Pharmacol. 2002, 50, 343-352]. A further marker which correlates with the expression of YB-1 is Topoisomerase II α. Insofar, rather than or in addition to determining YB-1 in the nucleus the expression of Topoisomerase II α or of any of the other markers described herein, can be used in a screening method to determine whether a patient may be treated with the adenoviruses according to the present invention with an expectation of success. A marker which can in principle be used similarly to the P-glycoprotein, is MRP. A further marker at least to the extent that the colorectal carcinoma cells or patients having a colorectal carcinoma are afflicted or may be/are identified, is PCN (proliferating cell nuclear antigen) (Hasan S. et al., Nature, 15, 387-391, 2001) as, for example, described in Shibao (Shibao K et al., Int. Cancer, 83, 732-737, 1999). Finally, at least for breast cancer and osteosarcoma cells the expression of MDR (multiple drug resistance) is a marker in the afore-described sense (Oda Y et al., Clin. Cancer Res., 4, 2273-2277). A further possible marker which can be used in accordance with the present invention, is p73 (Kamiya, M., Nakazatp, Y., J Neurooncology 59, 143-149 (2002); Stiewe et al., J. Biol. Chem., 278, 14230-14236, 2003).

It is a particular advantage of the present invention that also those patients may be subject to treatment using in accordance with the invention the adenoviruses described herein, which otherwise cannot be treated anymore in the medicinal-clinical sense and where thus a further treatment of the tumor diseases using the methods of the prior art is no longer possible with an expectation of success, in particular where the use of cytostatics and irradiation is no longer reasonably possible and cannot be successfully carried out any longer in the sense of influencing or reducing the tumor. Herein the term tumor refers in general also to any tumor or cancer disease which either inherently contains YB-1 in the nucleus of a cell, preferably independent of the cell cycle, or does so upon applying exogenous measures, as disclosed herein, and/or which contains deregulated YB-1.

Furthermore, the viruses described herein can be used, in principle, for the treatment of tumours.

The tumours which can in particular be treated by the viruses described herein are preferably those tumours which are selected from the group comprising tumours of the nervous system, ocular tumours, tumours of the skin, tumours of the soft tissue, gastrointestinal tumours, tumours of the respiratory system, tumour of the skeleton, tumours of the endocrine system, tumours of the female genital system, tumours of a mammary gland, tumours of the male genital system, tumours of the urinary outflow system, tumours of the haematopoietic system including mixed and embryonic tumours. It is within the present invention that these tumours are in particular resistant tumours as in particular defined herein.

The group of tumors of the nervous system preferably comprises:
1. Tumors of the skull as well as of the brain (intracranial), preferably astrocytoma, oligodendroglioma, meningioma, neuroblastoma, ganglioneuroma, ependymoma, schwannoglioma, neurofibroma, haemangioblastoma, lipoma, craniopharyngioma, teratoma and chondroma;
2. Tumors of the spinal cord and of the vertebral canal, preferably glioblastoma, meningioma, neuroblastoma, neurofibroma, osteosarcoma, chondrosarcoma, haemangiosarcoma, fibrosarcoma and multiple myeloma; and
3. Tumors of the peripheral nerves, preferably schwannoglioma, neurofibroma, neurofibrosarcoma and perineural fibroblastoma.

The group of the ocular tumors preferably comprises:
1. Tumors of the eyelids and of the lid glands, preferably adenoma, adenocarcinoma, papilloma, histiocytoma, mast cell tumor, basal-cell tumor, melanoma, squamous-cell carcinoma, fibroma and fibrosarcoma;
2. Tumors of the conjunctiva and of the nictitating membrane, preferably squamous-cell carcinoma, haemangioma, haemangiosarcoma, adenoma, adenocarcinoma, fibrosarcoma, melanoma and papilloma; and
3. Tumors of the orbita, the optic nerve and of the eyeball, preferably retinoblastoma, osteosarcoma, mast cell tumor, meningioma, reticular cell tumor, glioma, schwannoglioma, chondroma, adenocarcinoma, squamous-cell carcinoma, plasma cell tumor, lymphoma, rhabdomyosarcoma and melanoma.

The group of skin tumors preferably comprises:
Tumors of the histiocytoma, lipoma, fibrosarcoma, fibroma, mast cell tumor, malignant melanoma, papilloma, basal-cell tumor, keratoacanthoma, haemangiopericytoma, tumors of the hair follicles, tumors of the sweat glands, tumors of the sebaceous glands, haemangioma, haemangiosarcoma, lipoma, liposarcoma, malignant fibrous histiocytoma, plasmacytoma and lymphangioma.

The group of tumors of the soft-tissues preferably comprises:
Tumors of the alveolar soft-tissue sarcoma, epithelioid cell sarcoma, chondrosarcoma of the soft-tissue, osteosarcoma of the soft-tissues, Ewing's sarcoma of the soft-tissues, primitive neuroectodermal tumors (PNET), fibrosarcoma, fibroma, leiomyosarcoma, leiomyoma, liposarcoma, malignant fibrous histiocytoma, malignant haemangiopericytoma, haemangioma, haemangiosarcoma, malignant mesenchymoma, malignant peripheral nerve sheath tumor (MPNST, malignant schwannoglioma, malignant melanocytic schwannoglioma, rhabdomyosarcoma, synovial sarcoma, lymphangioma and lymphangiosarcoma.

The group of gastrointestinal tumors preferably comprises:
1. Tumors of the oral cavity and of the tongue, preferably squamous-cell carcinoma, fibrosarcoma, Merkel cell tumor, inductive fibroameloblastoma, fibroma, fibrosarcoma, viral papillomatosis, idiopathic papillomatosis, nasopharyngeal polyps, leiomyosarcoma, myoblastoma and mast cell tumor;
2. Tumors of the salivary glands, preferably adenocarcinoma;
3. Tumors of the oesophagus, preferably squamous-cell carcinoma, leiomyosarcoma, fibrosarcoma, osteosarcoma, Barrett carcinoma and paraoesophageal tumors;
4. Tumors of the exocrine pancreas, preferably adenocarcinoma; and
5. Tumors of the stomach, preferably adenocarcinoma, leiomyoma, leiomyosarcoma and fibrosarcoma.

The group of the tumors of the respiratory system preferably comprises:
1. Tumors of the nose and nasal cavity, of the larynx and of the trachea, preferably squamous-cell carcinoma, fibrosarcoma, fibroma, lymphosarcoma, lymphoma, haemangioma, haemangiosarcoma, melanoma, mast cell tumor, osteosarcoma, chondrosarcoma, oncocytoma (rhabdomyoma), adenocarcinoma and myoblastoma; and
2. Tumors of the lung, preferably squamous-cell carcinoma, fibrosarcoma, fibroma, lymphosarcoma, lymphoma, haemangioma, haemangiosarcoma, melanoma, mast cell tumor, osteosarcoma, chondrosarcoma, oncocytoma (rhabdomyoma), adenocarcinoma, myoblastoma, small-cell carcinoma, non-small cell carcinoma, bronchial adenocarcinoma, bronchoalveolar adenocarcinoma and alveolar adenocarcinoma.

The group of the skeleton tumors preferably comprises:
osteosarcoma, chondrosarcoma, parosteal osteosarcoma, haemangiosarcoma, synovial cell sarcoma, haemangiosarcoma, fibrosarcoma, malignant mesenchymoma, giant-cell tumor, osteoma and multilobular osteoma.

The group of the tumors of the endocrine system preferably comprises:
1. Tumors of the thyroid gland/parathyroid, preferably adenoma and adenocarcinoma;
2. Tumors of the suprarenal gland, preferably adenoma, adenocarcinoma and pheochromocytoma (medullosuprarenoma);
3. Tumors of the hypothalamus/hypophysis, preferably adenoma and adenocarcinoma;
4. Tumors of the endocrine pancreas, preferably insulinoma (beta cell tumor, APUDom) and Zollinger-Ellison syndrome (gastrin secernent tumor of the delta cells of the pancreas); and
5. multiple endocrine neoplasias (MEN) and chemodectoma.

The group of the tumors of the female sexual system tumors preferably comprises
1. Tumors of the ovaries, preferably adenoma, adenocarcinoma, cystadenoma, and undifferentiated carcinoma;
2. Tumors of the uterine, preferably leiomyoma, leiomyosarcoma, adenoma, adenocarcinoma, fibroma, fibrosarcoma and lipoma;
3. Tumors of the cervix, preferably adenocarcinoma, adenoma, leiomyosarcoma and leiomyoma;
4. Tumors of the vagina and vulva, preferably leiomyoma, leiomyosarcoma, fibroleiomyoma, fibroma, fibrosarcoma, polyps and squamous-cell carcinoma.

The group of tumors of the mammary glands preferably comprises:
fibroadenoma, adenoma, adenocarcinoma, mesenchymal tumora, carcinoma, carcinosarcoma.

The group of the tumors of the male sexual system preferably comprises:
1. Tumors of the testicles, preferably seminoma, interstitial-cell tumor and Sertoli cell tumor;
2. Tumors of the prostate, preferably adenocarcinoma, undifferentiated carcinoma, squamous-cell carcinoma, leiomyosarcoma and transitional cell carcinoma; and
3. Tumors of the penis and the external gentials, preferably mast cell tumor and squamous-cell carcinoma.

The group of tumors of the urinary outflow system preferably comprises:
1. Tumors of the kidney, preferably adenocarcinoma, transitional cell carcinoma (epithelial tumors), fibrosarcoma, chondrosarcoma (mesenchymal tumors), Wilm's tumor, nephroblastoma and embryonal nephroma (embryonal pluripotent blastoma);
2. Tumors of the ureter, preferably leiomyoma, leiomyosarcoma, fibropapilloma, transitional cell carcinoma;
3. Tumors of the urinary bladder, preferably transitional cell carcinoma, squamous-cell carcinoma, adenocarcinoma, botryoid (embryonal rhabdomyosarcoma), fibroma, fibrosarcoma, leiomyoma, leiomyosarcoma, papilloma and haemangiosarcoma; and
4. Tumors of the urethra, preferably transitional cell carcinoma, squamous-cell carcinoma and leiomyosarcoma.

The group of tumors of the haematopoietic system preferably comprises:
1. Lymphoma, lymphatic leukemia, non-lymphactic leukemia, myeloproliferative leukemia, Hodgkin's lymphoma, Non-Hodgkin's lymphoma.

The group of the mixed and embryonal tumors preferably comprises:
Haemangiosarcoma, thymoma and mesothelioma.

In a particularly preferred embodiment these tumors are selected from the group comprising breast cancer, ovary carcinoma, prostate carcinoma, osteosarcoma, glioblastoma, melanoma, small-cell lung carcinoma and colorectal carcinoma. Further tumors are those which are resistant as described herein, preferably those which are multiple resistant, particularly also those tumors of the group described above. Especially preferred tumors are also those selected from the group comprising breast tumors, bone tumors, stomach tumors, intestinal tumors, gallbladder tumors, pancreatic tumors, liver tumors, kidney tumors, brain tumors, ovary tumors, tumors of the skin and of cutaneous appendages, head/neck tumors, uterus tumors, synovial tumors, larynx tumors, oesophageal tumors, tongue tumors and prostate tumors. It is preferred that these tumors are, with regard to their manifestations, those which are disclosed herein.

Further tumours which can be treated using the viruses according to the present invention are leukaemia and metastatizing tumours, in particular metastatizing tumours of the previously recited tumours. Further tumours which may be treated in accordance with the present invention, are selected from the group comprising primary tumours, secondary tumours, tertiary tumours and metastatizing tumours. It is preferred if the tumours comprise at least one of the following features, namely that they have YB-1 in the nucleus independent of the cell cycle, regardless what the reason therefore is, and/or that they comprise deregulated YB-1. A further group of tumours which may be treated using the viruses according to the present invention, are all of the afore-mentioned tumours and tumours, respectively, which are described as being treatable by using the viruses according to the present invention, provided that they have one or several of the resistances disclosed herein.

It is further within the present invention that also such tumours can be treated using the viruses according to the present invention, which do neither contain YB-1 in the nucleus, preferably independent of the cell cycle, nor deregulated YB-1. This is realized in particular if the viruses themselves code for YB-1. For reasons of specific expression of YB-1 and thus of specific replication of the viruses, the expression of the viruses is put in a preferred embodiment under the control of a preferably highly regulated promoter. Such a promoter could be any promoter which can be activated in a specific manner so that the viruses can only replicate in the intended cells. Particularly preferred promoters are in particular tumour-specific promoters and tissue-specific promoters which are known to the ones skilled in the art. Furthermore, it is also possible to clone the sequence coding for YB-1 into the viral genome such that it is expressed by the adenoviral major late promoter (MLP). This promoter is mostly active after the start of the adenoviral replication (Tollefson A. E. et al., Journal of Virology 66, 3633-3642, 1992; Bauzon M. et al., Molecular Therapy 7, 526-534, 2003).

YB-1 belongs to the group of highly conserved factors which bind to an inverted CAAT sequence, the so-called Y-box. They may be active in a regulatory manner both at the level of transcription as well as translation (Wolffe, A. P. Trends in Cell Biology 8, 318-323, 1998).

The nucleic acid coding for YB-1 which, in an embodiment of the adenoviruses to be used in accordance with the present invention, is part of the adenoviruses, may also comprise a nucleic acid sequence mediating the transport of YB-1 into the nucleus. The nucleic acids, adenoviruses and adenoviral systems according to the invention as well as the adenoviruses known in the prior art such as, for example, Onyx-015, AdΔ24, dl922-947, E1Ad/01/07, CB016, dl 520 and the adenoviruses described in patent EP 0 931 830, can be used as such or in combination with these nucleic acids in accordance with the invention in connection therewith as adenoviruses and adenoviral systems and thus as the corresponding nucleic acids. Suitable nucleic acid sequences which mediate nucleus transport, are known to the ones skilled in the art and, for example, described in (Whittaker, G. R. et al., Virology, 246, 1-23, 1998; Friedberg, E. C., TIBS 17, 347, 1992; Jans, D. A. et al., Bioessays 2000 June; 22(6): 532-44; Yoneda, Y., J. Biocehm. (Tokyo) 1997 May; 121(5): 811-7; Boulikas, T., Crit. Rev. Eukaryot. Gene Expr. 1993; 3(3): 193-227; Lyons R H, Mol. Cell Biol., 7, 2451-2456, 1987). In connection with the nucleus transport mediating nucleic acid sequences, different principles can be used. One such principle may, for example, be that YB-1 is formed as a fusion protein together with a signal peptide and is introduced into the nucleus and that the replication of the adenoviruses according to the present invention thus occurs.

A further principle which may be realised in the design of the adenoviruses used in accordance with the invention, is that YB-1 can be provided with a transporter sequence which, preferably starting from synthesis in the cytoplasma, introduces YB-1 into the cell nucleus or which translocates YB-1 into the cell nucleus, and promotes viral replication there. An example for a particularly effective nucleic acid sequence mediating nucleus transport is the TAT sequence of HIV which is, among other suitable nucleic acid sequences of that type described in Efthymiadis, A., Briggs, L J, Jans, D A., JBC 273, 1623-1628, 1998. It is within the present invention that the adenoviruses which are used in accordance with the present invention, comprise nucleic acid sequences which code for peptides coding for nuclear transportation.

It is within the present invention that YB-1 is present in its full length, particularly in a form which corresponds to the wildtype of YB-1. It is within the present invention that YB-1 is used or present as a derivative, such as, e.g., in a shortened or truncated form. A YB-1 derivative as used or present within the present invention, is a YB-1 which is capable of binding to the E2-late promoter and thus activates gene expression of the adenoviral E2 region. Such derivatives particularly comprise the YB-1 derivatives disclosed herein. Further derivatives may be generated by deletion of single or several amino acids at the N-terminus, at the C-terminus or within the amino acid sequence. It is within the present invention that YB-1 fragments are also used as YB-1 proteins in the meaning of the present invention. Various YB-1 fragments are disclosed in the paper of Jürchott K et al. [JBC 2003, 278, 27988-27996] which are characterized by deletions in the C-terminus and the N terminus. The distribution of the various YB-1 fragments indicated that both the cold-shock domain (CSD) as well as the C-terminus are important for the cell cycle-regulated transport of YB-1 into the nucleus. It is thus within the present invention that a truncated YB-1 (which is also referred to herein as YB-1 protein) is migrating in a better way into the nucleus in combination with the expression of E1B55k and E4orf6 in accordance with the present invention and thus induces a stronger CPE without necessarily binding better to the E2-late promoter compared to native YB-1, whereby it cannot be excluded that also a truncated YB-1 is migrating better into the nucleus and exhibits both activities, i.e. induces CPE and binds to the E2-late promoter. Finally, such truncated YB-1 fragments can also better migrate into the nucleus and bind better to the E2-late promoter better without inducing a better CPE. It is also within the present invention that truncated YB-1 proteins or fragments comprise further sequences such as described herein in connection with the full length YB-1, in particular cellular localization signal sequences (NLS) and the like.

The invention is related in a further aspect to a method for the screening of patients which may be treated by using the viruses according to the present invention and/or by using the viruses and means and medicaments, respectively, described herein in accordance with the present invention, whereby the method comprises the following steps:
Analysing a sample of the tumor tissue and
Determining whether YB-1 is localised in the nucleus independent of the cell cycle, or whether the cell contains deregulated/overexpressed YB-1.

Instead of or in addition to YB-1 also the presence of the afore-described markers which represent an alternative to YB-1, can be assessed.

In an embodiment of the method according to the invention it is contemplated that the analysis of the tumor tissue occurs by means of an agent which is selected from the group comprising antibodies against YB-1, specifically binding peptides, aptamers against YB-1, spiegelmers against YB-1 as well as anticalines against YB-1. In principle, the same kind of agents can also be made and used, respectively, for the respective markers which represent an alternative to YB-1. The manufacture of antibodies, in particular monoclonal antibodies, is known to the ones skilled in the art. A further agent for specific detection of YB-1 or the markers are peptides which bind with a high affinity to their target structures, in the present case YB-1 or said markers. In the prior art methods are known such as, for example, phage-display, in order to generate such peptides. For such purpose, it is started from a peptide library whereby the individual peptides have a length of about 8 to 20 amino acids and the size of the library is about 10² to 10¹⁸, preferably 10⁸ to 10¹⁵ different peptides. A particular form of target molecule binding polypeptides are the so-called anticalines which are, for example, described in German patent application DE 197 42 706.

A further agent for specifically binding to YB-1 or the corresponding alternative markers disclosed herein and thus for the detection of a cell cycle independent localisation of YB-1 in the nucleus, are the so-called aptamers, i.e. D-nucleic acids, which, based on RNA or DNA, are present as either a single strand or a double strand and specifically bind to a target molecule. The generation of aptamers is, for example, described in European patent EP 0 533 838. A special embodiment of aptamers are the so-called aptazymes which, for example, are described by Piganeau, N. et al. (2000), Angew. Chem. Int. Ed., 39, no. 29, pages 4369-4373. They are a particular embodiment of aptamers insofar as they comprise apart from the aptamer moiety a ribozyme moiety and, upon binding or release of the target molecule binding to the aptamer moiety, the ribozyme moiety becomes catalyctically active and cleaves a nucleic acid substrate which goes along with generation of a signal.

A further form of the aptamers are the so-called spiegelmers, i.e. target molecule binding nucleic acids which consist of L-nucleic acids. The method for the generation of such spiegelmers is, for example, described in WO 98/08856.

The sample of the tumor tissue can be obtained by punctuation or surgery. The assessment whether YB-1 is located in the nucleus independent of the cell cycle is frequently done by the use of microscopic techniques and/or immunohistoanalysis, typically using the antibody or any of the further agents described above. Further methods for the detection of YB-1 in the nucleus and that its localisation there is independent of the cell cycle, are known to the one skilled in the art. For example, localisation of YB-1 can easily be detected when scanning tissue slices stained against YB-1. The frequency of YB-1 being in the nucleus is already an indication that the localisation in the nucleus is independent of the cell cycle. A further possibility for cell cycle independent detection of YB-1 in the nucleus is the staining against YB-1 and assessment whether YB-1 is localised in the nucleus and determining the phase of the cells. This and the detection of YB-1, respectively, however, can also be performed using the afore-mentioned agents directed against YB-1. The detection of the agents is done by procedures known to the persons skilled in the art. Because said agents are specifically directed against YB-1 and insofar do not bind to other structures within the sample to be analysed, particularly other structures of the cells, both the localisation of said agents by means of a suitable labelling of the agents and due to their specific binding to YB-1, both the localisation of YB-1 can be detected and assessed accordingly. Methods for the labelling of the agents are known to the ones skilled in the art.

It is within the present invention that the viruses described herein, whether they are the viruses according to the present invention, or whether they are the viruses to be used in accordance with the present invention, may also be used in connection with diseases, in particular tumor diseases and more preferably tumor diseases where at least part of the tumor cells exhibit a multiple resistance, in particular a multidrug resistance, whereby YB-1 is present in a deregulated form. This applies also to each and any of the other aspects as described herein in connection with cells and tumors, provided that they refer to the cells and diseases where YB-1 is present in the nucleus, preferably independent of the cell cycle.

Although the viruses in accordance with the present invention and as disclosed herein, are preferably adenoviruses the insights, methods and uses, nucleic acids, proteins, replication systems and the like are not limited to adenoviruses.

The aforementioned considerations, including any use as well as the generation of the adenoviruses and adenoviral systems, apply equally to the nucleic acids coding therefore and vice versa.

In connection with the present invention it is possible that the adenoviruses which are used in accordance with the present invention and the nucleic acids coding therefore, respectively, is any corresponding viral nucleic acid which result in a replication event per se or in combination with further nucleic acid sequences. It is possible, as explained herein, that by means of helper viruses the sequences and/or gene products are provided which are necessary for replication. To the extent it is referred to coding nucleic acid sequences and to the extent they are nucleic acid sequences which are known, it is within the present invention that not only the identical sequence, but also sequences derived therefrom, are used. Derived sequences are in particularly those sequences which still result in a gene product, either nucleic acid or a polypeptide having a function which corresponds to one or the function of the non-derived sequence. This can be determined by simple routine tests known to the one skilled in the art. An example for such derived nucleic acid sequences are nucleic acid sequences which code for the same gene product, in particularly the same amino acid sequence, however, due to the degeneracy of the genetic code, exhibit a different base sequence.

It is within the present invention that the viruses in accordance with the present invention are present as replication systems with or without helper viruses.

It is further within the present invention that in case of such adenoviral replication system according to the invention the adenoviral nucleic acid and/or the nucleic acid of the helper virus is present as a replicable vector.

It is further within the present invention that the nucleic acid(s) coding for the adenoviruses which are used in accordance with the present invention, is/are present in a vector, preferably an expression vector and that this expression vector is used in accordance with the present invention.

In a further aspect the present invention is also related to a vector group comprising at least two vectors, whereby the vector group in its entirety comprises a viral replication system as described herein, and the vector group is used in accordance with the present invention. It is within the invention that each of the components of the viral replication system is present on a separate vector, preferably an expression vector.

Finally, the present invention is related in a further aspect to the use of a cell which contains one or several of the nucleic acids which code for the viruses described herein and which are to be used in accordance with the present invention, and/or a corresponding adenoviral replication system and/or a corresponding vector and/or a vector group according to the invention, for the very same purpose as described herein for the various viruses.

The above described constructs of viruses and in particular their nucleic acids and the nucleic acids coding therefor, respectively, may also be introduced in a multipartite form into a cell, preferably a tumour cell, whereby, due to the presence of the various individual components, these components act together as if the individual components were derived from a single nucleic acid and a single or several viruses, respectively.

The nucleic acids which are used in accordance with the invention and which code for viruses, viral systems or parts thereof, may also be present as vectors. Preferably these vectors are viral vectors. In case the nucleic acids comprise viral nucleic acids, preferably the virus particle is the vector. It is, however, also within the present invention that said nucleic acids are present in a plasmid vector. In each case the vector comprises elements which allow for and control the propagation of inserted nucleic acid, i.e. replication and the optional expression of the inserted nucleic acid. Suitable vectors, preferably expression vectors, and respective elements are known to the ones skilled in the art and, for example, described in Grunhaus, A., Horwitz, M. S., 1994, Adenoviruses as cloning vectors. In Rice, C., editor, Seminars in Virology, London: Saunders Scientific Publications.

The above described embodiment where the various elements of said nucleic acids are not necessarily contained in only one vector, takes into consideration the aspect of the invention related to the vector group. A vector group comprises accordingly at least two vectors. Otherwise the general statements made herein in relation to vectors are applicable to the vectors and the vector group, respectively.

The viruses used in accordance with the present invention are characterised by the various nucleic acids and gene products, respectively, disclosed herein and may otherwise comprise all those elements known to the person skilled in the art and as is the case in particular with wildtype adenoviruses (Shenk, T.: Adenoviridae: The virus and their replication. Fields Virology, vol. 3, editors Fields, B. N., Knipe, D. M., Howley, P. M. et al., Lippincott-Raven Publishers, Philadelphia, 1996, chapter 67).

In a further aspect the present invention is related to a method for the treatment of tumor diseases comprising the administration of a virus according to the present invention, such nucleic acid, vectors, replication systems, medicaments or pharmaceutical compositions. The tumor disease is one as disclosed herein. The patient is in need of such treatment and is preferably a patient selected from the groups of patients disclosed herein.

It is within the present invention that, if not indicated to the contrary, the features and embodiments disclosed for the respective viruses, nucleic acids, vectors, replication systems, medicaments and pharmaceutical compositions, each according to the invention, and those of the viruses, nucleic acids, vectors, replication systems, medicaments and pharmaceutical compositions to be used in accordance with the invention, are also applicable to each and any of the other aspects of the present invention and vice versa.

In the following the present invention shall be further illustrated by reference to the figures and examples from which new features, embodiments and advantages may be taken.

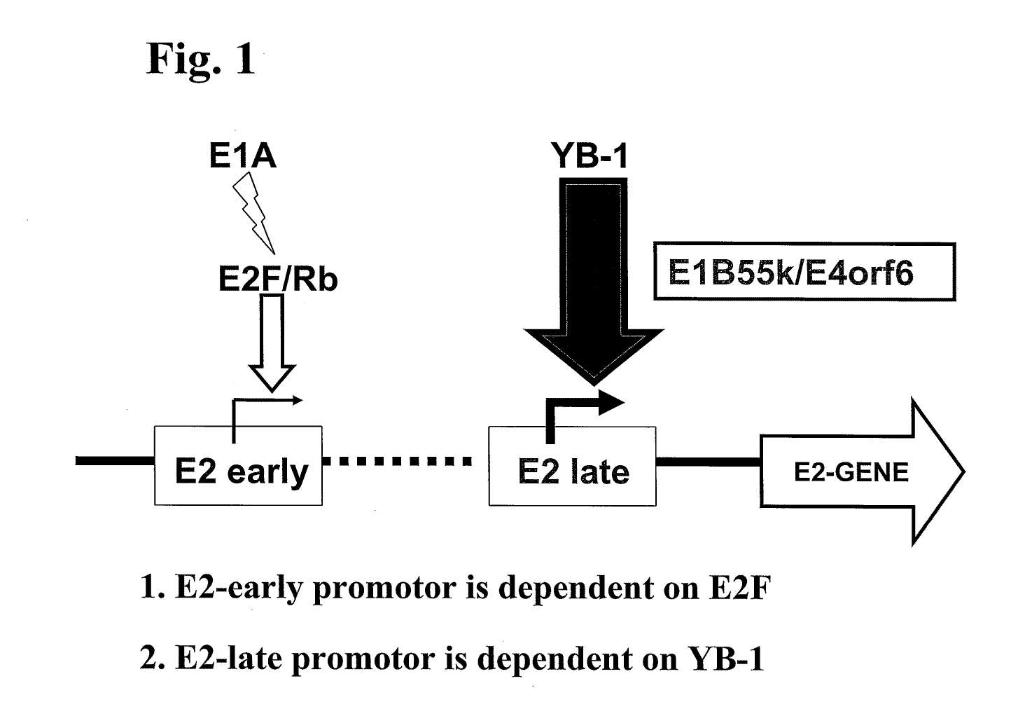

FIG. 1 is a schematic representation of the regulation of the E2 region of adenoviruses by the promoters E2 late and E2 early by means of E2F and YB-1;

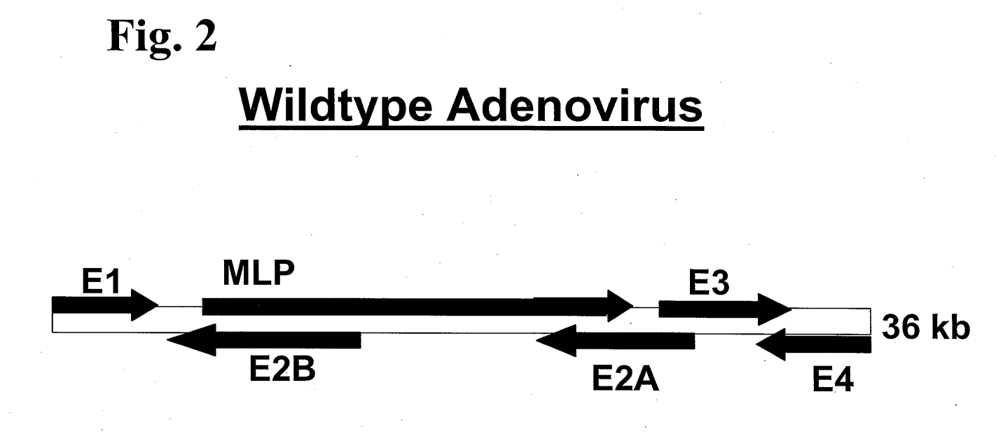

FIG. 2 is a schematic representation of the design of the adenovirus of the wildtype;

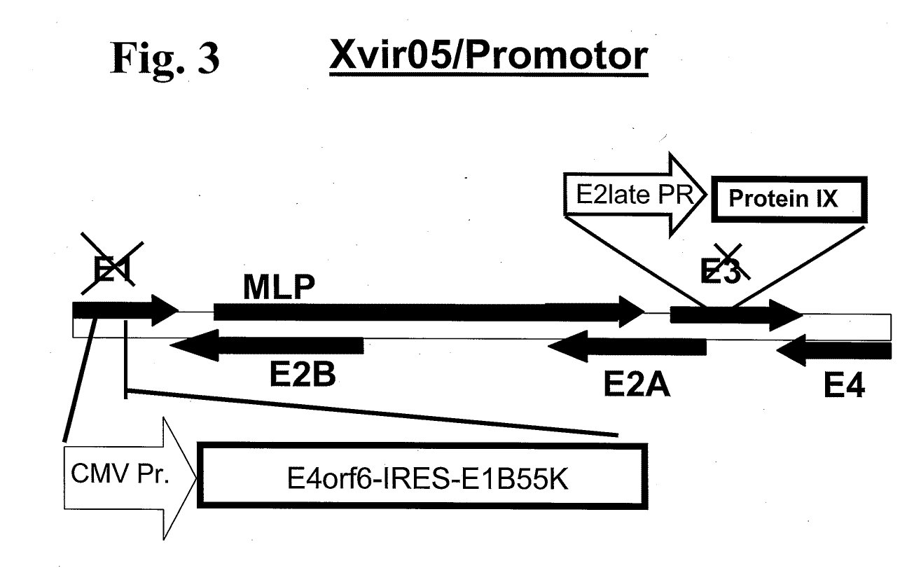

FIG. 3 is a schematic representation of the adenovirus Xvir 05/promoter according to the invention which expression protein IX under the control of the E2 late promoter;

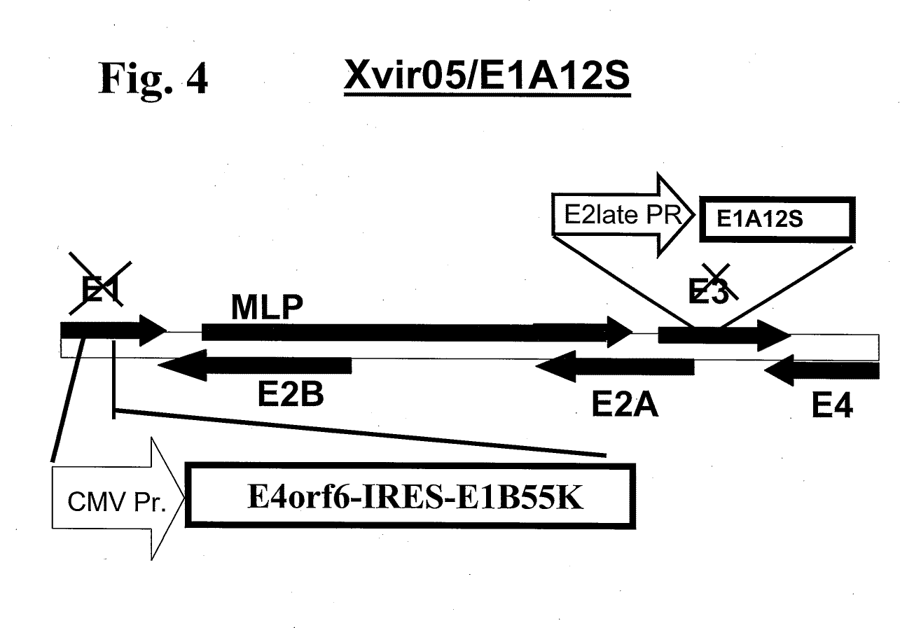

FIG. 4 is a schematic representation of the adenovirus Xvir 05/E1A12S of the invention which expresses protein IX as part of the E1B55K reading frame under the control of E1A12S;

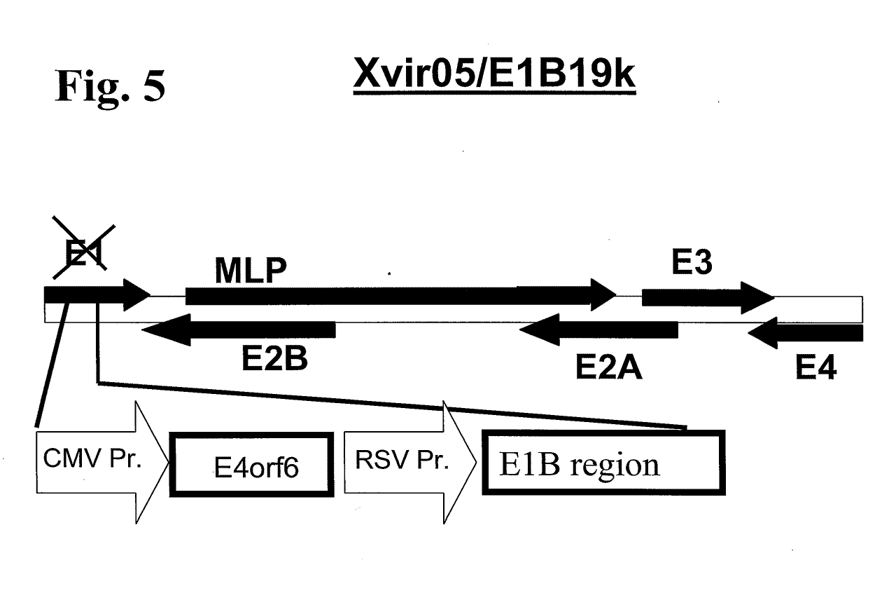

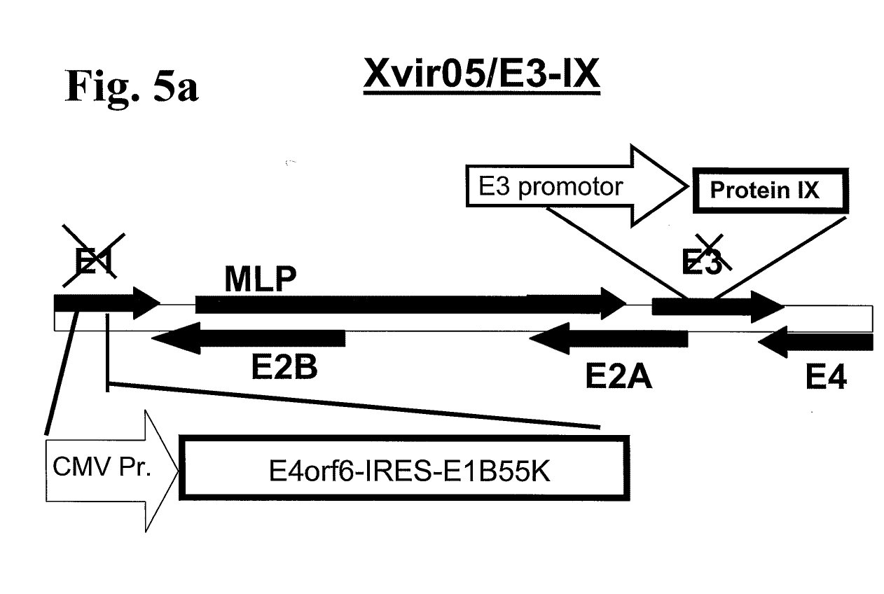

FIG. 5 is a schematic representation of an adenovirus Xvir 05/E1B19K according to the invention which expresses protein IX under the control of E1B19K;

FIG. 5a is a schematic representation of the adenovirus Xvir 05/E3-IX according to the invention which expresses protein IX under the control of the E3 promoter;

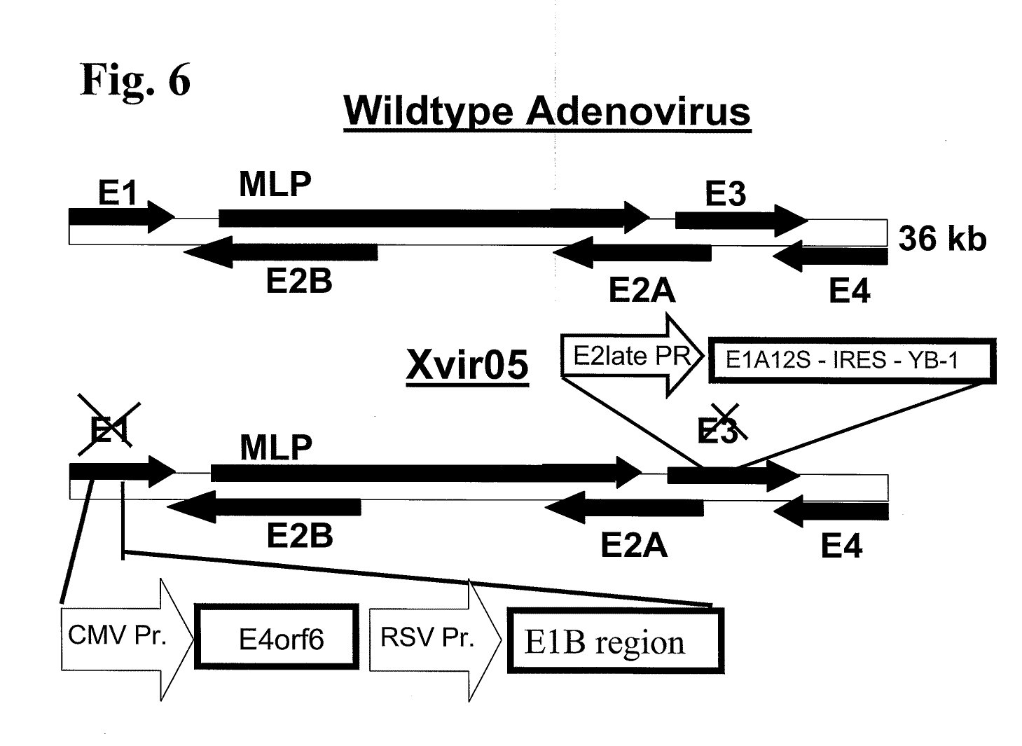

FIG. 6 is a schematic representation of the wildtype adenovirus and the adenovirus Xvir 05 according to the invention which is an embodiment of the virus Xvir 05/E1B19K;

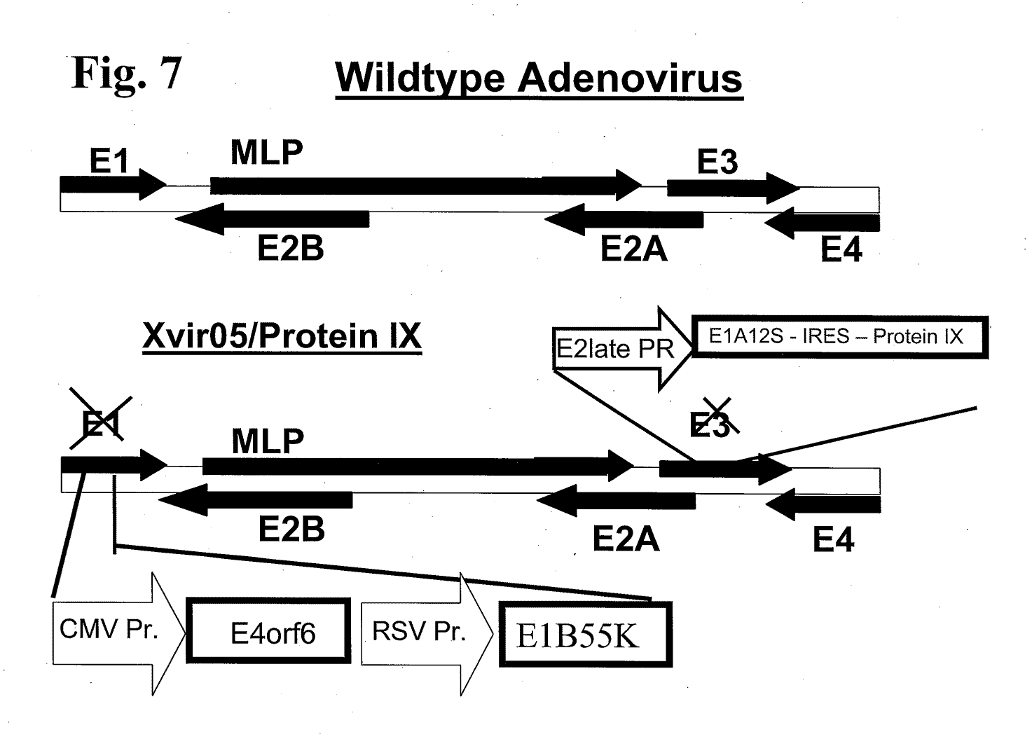

FIG. 7 is a schematic representation of the wildtype adenovirus and the adenovirus Xvir 05/protein IX according to the invention which is an embodiment of the virus Xvir 05/E1A12S;

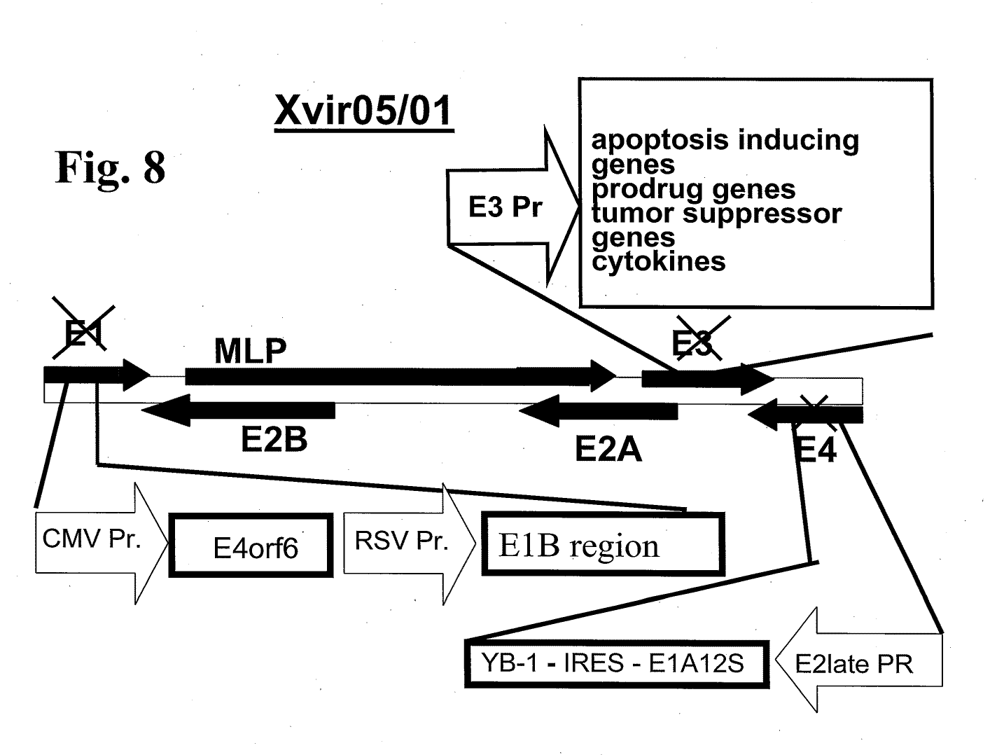

FIG. 8 is a schematic representation of the wildtype adenovirus and the adenovirus Xvir 05/01 according to the invention which is an embodiment of the virus Xvir 05/protein IX;

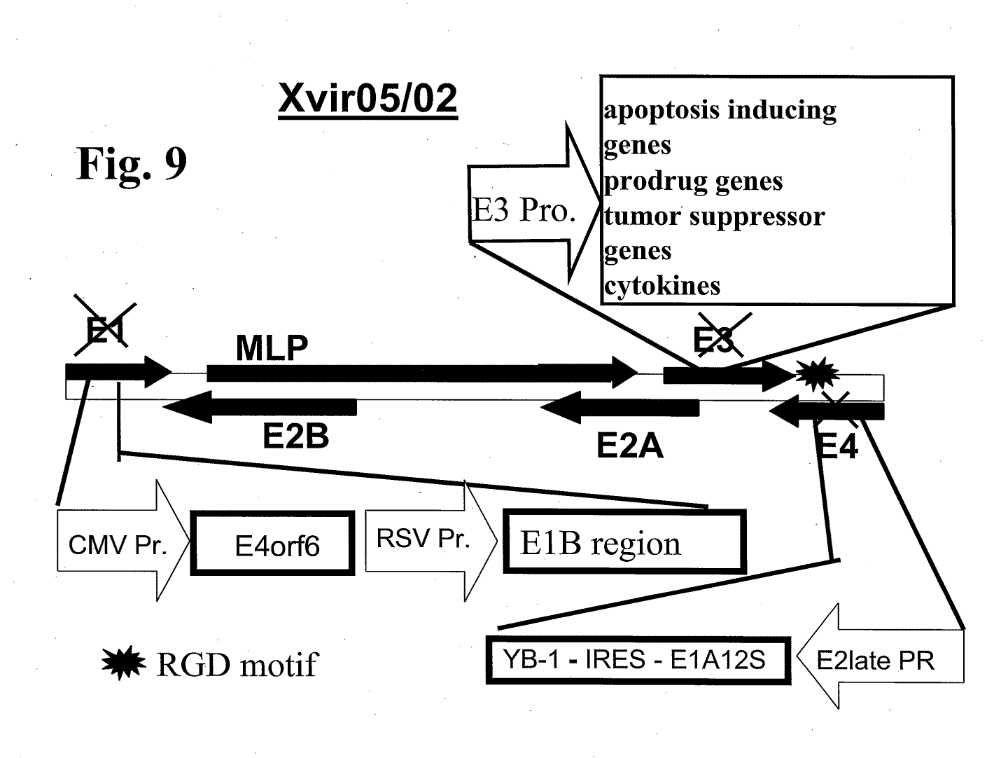

FIG. 9 is a schematic representation of the wildtype adenovirus and the adenovirus Xvir 05/02 according to the invention which is a further embodiment of the virus Xvir 05/protein IX;

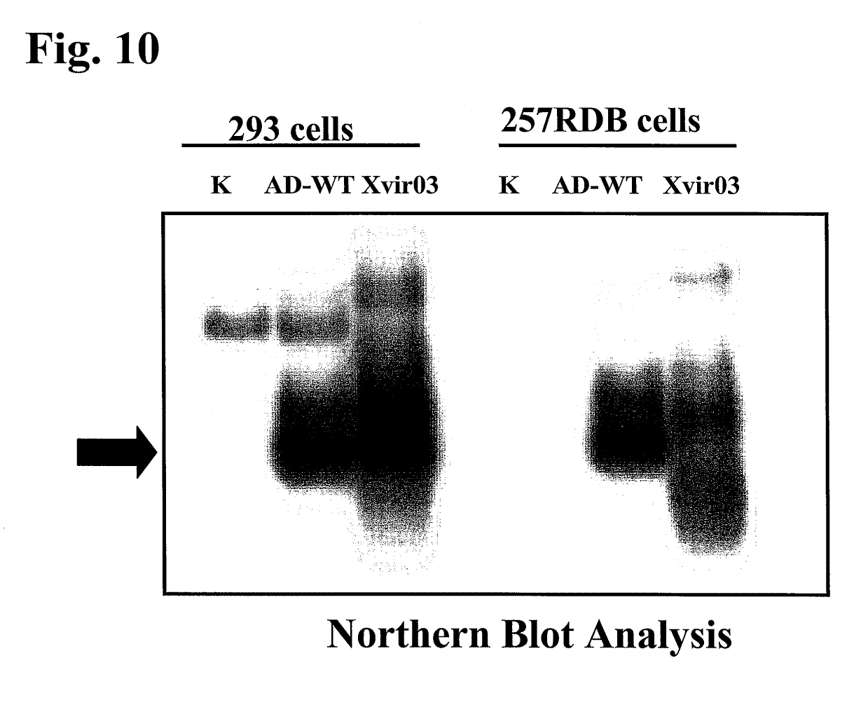

FIG. 10 is the result of a Northern blot analysis for the detection of protein IX; and

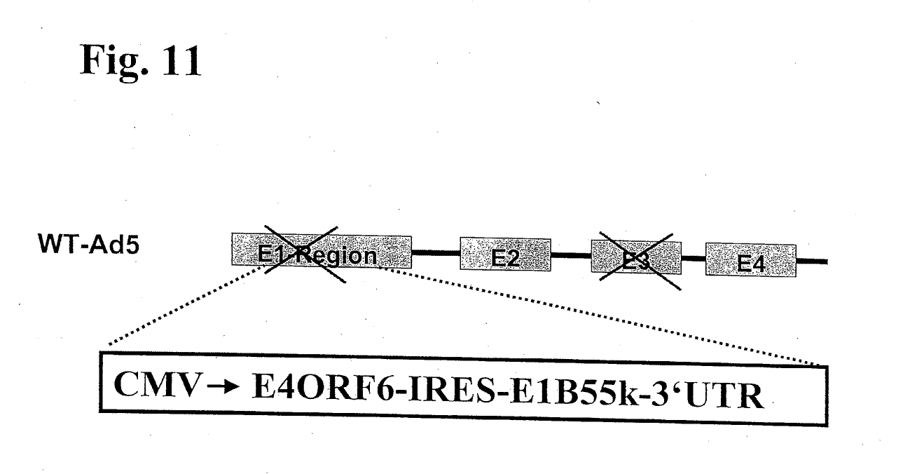

FIG. 11 is a schematic representation of the design of the oncolytic adenovirus Xvir03-3′UTR.

FIG. 1 is a schematic representation of the regulation of the E2 region of adenovirus by the promoters E2-late and E2-early by means of E2F and YB-1. In FIG. 1 the involved promoters, namely the E2-early and E2-late promoters, are represented with regard to the binding and activation, respectively, by means of E2F and YB-1. The wildtype E1A protein is interfering with the binding of E2F to retinoblastoma protein Rb. The thus released E2F is binding to the E2 early promoter and thus induces adenoviral replication. After 8-12 h a so-called switch occurs to the E2-late promoter. This is only possible upon the translocation of YB-1 from the cytoplasma into the nucleus. After nuclear translocation YB-1 activates the E2 gene expression through the binding to the E2-late promoter.

The binding mechanism of E2F/RB and the E1A mediated release of E2F is substantially different from the mechanism underlying the present invention. The release of E2F from the Rb protein is not, as assumed in the prior art, an important, not to say the decisive step of adenoviral replication, but the nuclear localisation of the human transcription factor YB-1. This transcription factor is present in normal cells over the major part of the cell cycle only in the cytoplasm. After infection with an adenovirus it is induced in the nucleus under certain conditions or is already present in the nucleus in distinct cellular systems such as distinct tumor diseases, i.e. including, but not limited to, breast cancer, ovarian carcinoma, prostate cancer, osteosarcoma, glioblastoma, melanoma, small-cell carcinoma of the lung and colorectal cancer.

### EXAMPLE 1

### Design of Various Protein IX Expressing Adenoviruses

Starting from the design depicted in FIG. 2 of the viral nucleic acid of the wildtype adenovirus the various design principles disclosed herein for the expression of protein IX by adenoviruses which replicate in a YB-1 dependent manner, are realised and are depicted in FIGS. 3, 4, 5 and 5a. All designs have in common that they are E1A13S-minus and E1A12S-minus in the sense that they are not controlled by the natural and the E1A promoter present in the wildtype, respectively.

The adenovirus Xvir 05/promoter as depicted in FIG. 3 is additionally E1B19K-minus and protein IX-minus in the sense that protein IX is not contained in the regulatory context as existing in the wildtype, and protein IX is not expressed. Rather, the expression is controlled by the E2-late promoter. Protein IX is cloned into the E3 region, however, may, in principle, also be cloned into the E4 region. The genes for E2A, E2B, E4 and MLP are still present and can also be expressed. The transporter consisting of E4orf6 and E1B55K is formed by the cassette E4orf6-IRES-E1B55K which is under the control of the CMV promoter. The respective cassette has been cloned into the E1 region, however, could also be cloned into other regions such as, for example, the E3 or E4 region.

The adenovirus of the adenovirus Xvir05/E1A12S as depicted in FIG. 4 is additionally E1B19K-minus and protein IX-minus in the sense that protein IX is not present in the regulatory context existing in the wildtype and protein IX is not expressed. Rather, the expression is caused by E1A12S which is controlled by the E2-late promoter which results in the activation of the reading frame of protein IX which is contained in the E1B55K coding region. Protein E1A12S has been cloned into the E3 region, however, may also be cloned into the E4 region. The genes for E2A, E2B, E4 and MLP are still present and can also be expressed. The transporter consisting of E4orf6 and E1B55K is formed by the cassette E4orf6-IRES-E1B55K which is under the control of the CMV promoter. The respective cassette has been cloned into the E1 region, however, could also be cloned into different regions such as into the E3 or E4 region.

The adenovirus of adenovirus Xvir 05/E1B19K as depicted in FIG. 5 is additionally E1B19K-minus and protein IX-minus in the sense that protein IX is not contained in the regulatory context as existing in the wildtype. Rather the expression is controlled by protein E1B19K which is expressed under the control of the CMV promoter and which allows the expression of the reading frame of protein IX which is contained in the E1B55K reading frame. The genes for E2A, E2B, E3, E4 and MLP are still present and can also be expressed. The transporter consisting of E4orf6 and E1B55K is formed by the cassette E4orf6-RSV-promoter-E1B region which is under the control of the CMV promoter. The respective cassette has been cloned into the E1 region, however, could also be cloned into different regions such as, for example, the E3 or E4 region.

The adenovirus of adenovirus Xvir05/E3-IX as depicted in FIG. 5a is additionally E1B19K-minus and protein IX-minus in the sense that protein E1B19K is not contained in the regulatory context of the wildtype and that protein IX is not expressed. Rather the expression is controlled by the natural E3 promoter. The genes for E2A, E2B, E4 and MLP are still present and can also be expressed. The transporter consisting of E4orf6 and E1B55K is formed by the cassette E4orf6-IRES-E1B55K which is under the control of the CMV promoter. The respective cassette has been cloned into the E1 region, however, could also be cloned into other regions such as the E3 or E4 region.

FIGS. 6 to 9 represent further embodiments of the adenoviruses of the invention.

The virus depicted in FIG. 6 is a further development of adenovirus Xvir 05/E1B19K as depicted in FIG. 5. In addition to Xvir05/E1B19K this virus comprises a cassette which is under the control of the E2-late promoter and which comprises E1A12S and YB-1 and a nucleic acid, respectively, coding each and individually therefor, whereby both reading frames are separated from each other by an IRES. In an embodiment the YB-1 coding nucleic acid is not contained in the cassette. The nucleic acid for YB-1 expressed by the virus results in a more pronounced replication in cells with deregulated YB-1.

The adenovirus depicted in FIG. 8 is a further development of the adenovirus depicted in FIG. 6, whereby the cassette which is under the control of the E2-late promoter comprises E1A12S and YB-1 and a nucleic acid, respectively, coding each and individually therefor, which is cloned into the E4 region and various transgenes are cloned into the E3 region under the control of the E3 promoter such as, for example, apoptosis-inducing genes, prodrug genes, siRNA, tumor suppressor genes and cytokines. Alternatively, the various transgenes disclosed herein may be cloned into this region.

Finally, the adenovirus of the invention as depicted in FIG. 9 is a further development of the adenovirus depicted in FIG. 8, whereby here additionally the RGD motif which is advantageous for the targeting of the viruses, has been incorporated by cloning. It can be found in the adenoviral genome in the fibre protein approximately at positions 32675-32685. These variations of the specific position details is caused by the fact that the sequences of the wildtype adenovirus are different and have different lengths in the various data banks or data bank entries.

The adenovirus of the invention depicted in FIG. 7 is based on the virus depicted in FIG. 3. In contrast thereto, this adenovirus, however, does not comprise a cassette consisting of E4orf6 and E1B55K, but both are controlled by separate promoters, namely the CMV promoter and the RSV promoter. The cloning had been made into the E1 region. Additionally, the adenovirus comprises apart from the nucleic acid coding for E1A12S which is under the control of the E2-late promoter, still a nucleic acid, which codes for protein IX, which is separated from the one of E1A12S by an IRES. Also this cassette could in principle be designed without the nucleic acid coding for protein IX. In a further embodiment the cassette is cloned into the E4 region. Finally, also this virus could comprise in the E3 or the E4 region transgenes as described in connection with the virus depicted in FIG. 8. In a further embodiment of this adenovirus the RGD motif is contained.

### EXAMPLE 2

### Detection of Protein IX Expression

This experiment was carried out in order to confirm the relevance of the expression of protein IX for effective particle formation in YB-1 mediated replication. For such purpose the oncolytic, YB-1 dependent replicating adenovirus Xvir 03-03′UTR was used which is described in the prior art and which is depicted in FIG. 11.

When carrying out the experiment it was proceeded as follows: Per 10 cm dish 10⁶ 293 and 257RDB cells were plated. At the next day the cells were, as depicted in FIG. 11, either not infected (K), infected with the wildtype adenovirus or with Xvir03. The infection was made in 1.5 ml serum-free DMEM medium for 1 h at 37° C. Subsequently the infection medium was removed and replaced by 10 ml whole medium (10% FCS/DMEM). After 24-48 h RNA was isolated. Subsequently, a Northern blot analysis was performed. For such purpose, each 10 μg RNA were separated by electrophoresis in an agarose gel containing 3% formaldehyde, subsequently blotted onto a nylon membrane and hybridised against a 386 bp probe. As probe which was generated by PCR, a P32 labelled probe was used targeting protein IX. The following primer was used for the PCR: 5′-TATTTGACAACGCG; 5′-TTTTAAACCGCATTGGG. The position of the probe in the adenovirus genome of the wildtype is between position 3648 and 4033. The virus used is Xvir 03 which does not show expression of protein IX.

The result of this experiment is depicted in FIG. 10.

As may be taken from FIG. 11, virus Xvir03-03′UTR shows a reduced expression in tumor cells 257RDB compared to wildtype adenovirus. In 293 cells which express E1A and E1B proteins, among others also the E1B19K protein, sufficient protein IX is expressed.

### EXAMPLE 3

### Structure of Recombinant Adenoviruses Xvir05, Xvor05/Protein IX, Xvir05/01 and Xvir05/02

In vector Xvir05 the expression of, among others, the viral proteins E4orf6 and E1B55k is provided by the expression cassettes CMV-E4orf6 and RSV-E1B-region. This results in translocation of YB-1 into the nucleus. The E1A12S gene product, as well as the YB-1 gene product, under the control of the E2-late promoter, additionally promote viral replication. Additionally, the virus is capable of inhibiting the expression of the ABC transporters MRP and MDR1. Additionally, the proteins E1B19K and protein IX are expressed as part of the cassette RSV-E1B region.

The vector Xvir05-protein IX is a further vector development. There, the expression of the adenoviral protein IX which is present in the expression cassette E2late-E1A12S-IRES-protein IX is ensured. The vector does not contain the whole E1B region, but only the open reading frame of E1B55k.

The complete E1B region, i.e. E1B19k, E1B55k and protein IX are controlled by a viral, non-adenoviral promoter in case of vector Xvir05/01, such as the RSV promoter. The expression cassette E2-late-E1A12S-IRES-YB-1 is present in the E4 region. Thus specific therapeutic transgenes can be cloned into the E3 region. The E3 deletion is such that the adenoviral ADP protein “adenoviral death protein” can still be expressed. Additionally, the expression of E1A12S and E1B19k results in the expression of protein IX.

The vector Xvir05/02 additionally comprises an RGD motif in the H loop of the fibre knob in order to provide for better infection rates.

The preparation of the virus was as follows:

Modification of the Rescue Plasmid pAdEASY (Qbiogene)
Use of the Shuttle Vector pShuttle-AdEASY for the Preparation of a ΔE3E4 Shuttle Vector

First a CMV promoter was introduced into the available vector pShuttle-AdEASY and a bovine growth hormone polyadenylation signal cloned into it. For such purpose the plasmid was digested with EcoRI, the ends made blunt-ended by filling with T4 polymerase and dNTPs, the backbone dephosphorylated and the two resulting cleavage products religated. By this procedure the restriction recognition sequences for EcoRI were destroyed. The plasmid thus obtained was referred to as pShuttle(-EcoRI)-AdEASY.

Subsequently, the cassette CMV-MCS-polyA was excised from the pShuttle of Clontech by using MfeI and EcoRI, the ends made blunt-ended and cloned into the vector pShuttle(-EcoRI)-AdEASY which was linearised with XbaI, made blunt-ended and dephosphorylated for such purpose. The plasmid CMV-MCS-PolyA-pShuttle-AdEASY was thus generated.

For the manipulation of the E3 and E4 region the ΔE3E4 region of the plasmid pAdEASY was cloned with SpeI and PacI into plasmid CMV-MCS-PolyA-pShuttle-AdEASY and thus the plasmid ΔE3E4 pShuttle-AdEASY prepared. By restriction with NdeI and religation one of the two NdeI cleavage sites was deleted and thus also the multiple cloning site from the plasmid. By this procedure plasmid ΔE3E4-pShuttle(-NdeI)-AdEASY was obtained.

### E4 Manipulation

In order to provide space for potential therapeutic transgenes and in order to avoid an undesired homologous recombination, the E4 region in plasmid ΔE3E4-pShuttle(-NdeI)-AdEASY was specifically deleted. For such purpose the E4orf6 region was truncated by about 634 bp by means of cleavage with PstI and religation=ΔE3E4ΔORF6-pShuttle(-NdeI)-AdEASY. The respective deletions can be made in other systems for the generation of recombinant adenoviruses by a person skilled in the art.

Cloning of the RGD Motif in ΔE3E4ΔORF6-pShuttle(-NdeI)-AdEASY

For an improved infectivity and referring to Dmitriev et al. 1998 (An Adenovirus Vector with Genetically Modified Fibers Demonstrates Expanded Tropism via Utilization of a Coxsackievirus and Adenovirus Receptor-Independent Cell Entry Mechanism) the HI loop of the fibre knob domain was modified: The respective region was amplified using the primers RGD-Hpa fw (5′-GAGgttaacCTAAGCACTGCCAAG-3′), RGD-EcoRV rev (5′-CATAGAGTATGCAGATATCGTTAGTGTTACAGGTTTAGTTTTG-3′) as well as RGD-EcoRV fw (5′-GTAACACTAACGATATCTGCATACTCTATGTCATTTTCATGG-3′) and RGD-BfrI rev (5′-CAGCGACATGAActtaagTGAGCTGC-3′) and thus an EcoRV cleavage site generated. Into this cleavage site paired oligonucleotides were cloned which coded for Arg-Gly-Asp (RGD) peptide: RGD-Oligo 1 (5′-CACACTAAACGGTACACAGGAAACAGGAGACACAACTTGTGACTGCCGCGGAGA CTGTTTCTGCCC-3′) and RGD-Oligo 2 (5′-GGGCAGAAACAG TCTCCGCGGCAGTCA CAAGTTGTGTCTCCTGTTTCCTGTGTACCGTTTAGTGTG-3′). By cloning into the HpaI and BfrI cleavage site in the ΔE3E4ΔORF6-pShuttle(-NdeI)-AdEASY ΔE3-RGD-E4ΔORF6-pShuttle(-NdeI)-AdEASY was generated. The RGD motif is present in the HI loop of the fibre knob domain.

Cloning of the E3a Region into the ΔE3 Region of ΔE3RGD-E4ΔORF6-pShuttle(-NdeI)-AdEASY.

For such purpose the vector pcDNA3.1(+) of Invitrogen was digested with BglII and BamHI, whereby the CMV promoter was removed and the vector religated (pcDNA3.1(+) without CMV=oCMV). To the SpeI and XhoI restriction sites of the pcDNA3.1(+) oCMV vector the 2709 bp fragment which was dissected with SpeI (27083 bp) and XhoI (29792 bp) from the wildtype virus DNA, was cloned into (pcDNA3.1(+) oCMV/E3aXhoI). Alternatively, one may cut at the 3′ end with HpaI (30570 bp) rather than with XhoI. For such purpose the vector pcDNA3.1 (+) oCMV is then digested with SpeI and EcoRV and the adenoviral SpeI-HpaI-fragment cloned therein (pcDNA3.1(+) oCMV/E3aHpaI). A further option is provided by the 2718 bp EcoRI fragment from the adenovirus wildtype DNA (positions 27332 bp and 30050 bp) which is cloned into pcDNA3.1 (+) oCMV which has been opened using EcoRI (pcDNA3.1(+) oCMV/E3aEcoRI).

Using pcDNA3.1(+) oCMV/E3a the E3a region could be cloned into the vector ΔE3RGD-E4ΔORF6-pShuttle(-NdeI)-AdEASY: The shuttle vector ΔE3RGD-E4ΔORF6-pShuttle(-NdeI)-AdEASY was digested with NheI for such purpose, the ends made blunt-ended and further digested with SpeI. The insert from pcDNA3.1(+) oCMV/E3aXhoI was cloned into this site: The plasmid was digested with XhoI for such purpose, the ends made blunt-ended and further digested with SpeI. The fragment thus excised was cloned into the previously cut open plasmid ΔE3RGD-E4ΔORF6-pShuttle(-NdeI)-AdEASY.

The fragments SpeI-HpaI (position 27083 bp to 30570 bp) and EcoRI (position 27332 bp to 30050 bp) may be prepared in a similar manner from the respective pcDNA3.1(+) oCMV/E3a constructs and cloned.

Alternatively, the E3a region may be amplified by PCR using the primers E3a forward (SpeI) 5′-CTTAAGGACTAGTTTCGCGC-3′ and E3a reverse (XhoI, NheI) 5′-CAAGCTAGCTCGAGGAATCATG-3′ using the adenovirus type 5 wildtype DNA as template. With the E3a reverse primer a NheI cleavage site is generated. The amplificate is restricted with SpeI and NheI and cloned into the vector ΔE3RGD-E4ΔORF6-pShuttle(-NdeI)-AdEASY also digested with SpeI and NheI.

### For the SpeI-HpaI Fragment

Alternatively, the E3a region can be amplified by PCR using the primers E3a forward (SpeI) 5′-CTTAAGGACTAGTTTCGCGC-3′ and E3a reverse (HpaI, NheI) 5′-CACGCTAGCAAGTTAACCATGTCTTGG-3′ using the adenovirus type 5 wildtype DNA as template. Using the E3a reverse primer an NheI cleavage site is thus generated. The amplificate is restricted with SpeI and NheI and cloned into the vector ΔE3RGD-E4ΔORF6-pShuttle(-NdeI)-AdEASY which is also opened by SpeI and NheI.

### For the EcoRI Fragment

Alternatively, the E3a region can be amplified by PCR using the primers E3a forward (EcoRI) 5′-GAAACCGAATTCTCTTGGAAC-3′ and E3a reverse (NheI, EcoRI) 5′-GAATTCTAGCTAGCTCAGCTATAG-3′ using the adenovirus type 5 wildtype DNA as template. Using the E3a reverse primer an NheI cleavage site is thus generated. The amplificate is restricted with EcoRI and NheI and cloned into the vector ΔE3RGD-E4ΔORF6-pShuttle(-NdeI)-AdEASY also cut open with EcoRI and NheI.

The cloning of the E3a region from pcDNA3.1(+) oCMV/E3a in ΔE3RGD-E4ΔORF6-pShuttle(-NdeI)-AdEASY E3aΔE3RGD-E4ΔORF6-pShuttle(-NdeI)-AdEASY was generated.

The thus cloned region comprises the E3 region until after the open reading frame for the E3 ADP (position 29772 bp) and thus the E3 promoter, the complete E3A region with the polyadenylation signal, the transcription start and the open reading frame for 12.5 K, E3 6.7 K, E3 gp19 K and E3 ADP.

The E3 region is, compared to the adenovirus type 5 DNA sequence, in case of SpeI-XhoI cloning deleted from position 29796 to 31509 bp (=1713 bp).

Further deletions between the E3 promoter and the open reading frame for the ADP are possible with plasmid pcDNA3.1(+) oCMV/E3a: By further restrictions between position 27596 bp and 29355 bp, for example with EcoRII, BsiWI, DraI, MunI, the open reading frames for 6.7 K and gp19 K which are present in between, may be removed and thus provided up to 1.8 kb of additional space for the incorporation of further transgenes. By a respective restriction the above noted E3A amplificates can also be truncated and be cloned as previously described.

### Cloning of the Second Expression Cassette E1a 12S Under the Control of the E2 Late Promoter.

First, the E2 late promoter was cloned as paired oligonucleotide (upper primer 5′-TCGAGCTCCGCATTTGGCGGGCGGGATTGGTCTTCGTAGAACCTAATCTCGTGGG CGTGGTAGTCCTCAGGTACAAAT-3′ and lower primer 5′-AGCTTATTTGTACCTGAGGACTACCACGCCCACGAGATTAGGTTCTACGAAGACC AATCCCGCCCGCCAAATGCGGAGC-3′ in the HindIII and BglII cleavage site of the pGL3-enhancer plasmid of Promega (pGL3-E2Late).

Subsequently, the luciferase gene was excised with NcoI and XbaI, the ends made blunt-ended and T ends added. At the thus opened site the transgene E1A 12S which was amplified by RT-PCR using the primers E1a 12S forward 5′-ATGGCCGCCAGTCTTTTG-3′ and E1a 12S reverse 5′-TTATGGCCTGGGGCGTTTAC-3′, was introduced by TA cloning.

By doing so, the cassette contains the E2-late promoter, the open reading frame E1a-12S and the SV-40 late polyadenylation signal of the vector pGL3.

This cassette was excised with PvuI and ClaI, the ends made blunt-ended and can now alternatively be cloned into the E3a region which was deleted by EcoRII, BsiWI, DraI, MunI (after removal of the open reading frames for E3 6.7 K and gp19 K as above) or into the deletion of the E4ORF6, for example into the blunt-ended and phosphorylated BfrI cleavage site.

The thus generated construct is E3a/E2Late-E1a-12S/ΔE3RGD-E4ΔORF6-pShuttle(-NdeI)-AdEASY or E3aΔE3RGD-E4ΔORF6/E2Late-E1a-12S-pShuttle(-NdeI)-AdEASY.

Cloning of the Second Expression Cassette E1a 12S with YB-1 Under the Control of the E2Late Promoter

The amplificates E1a 12S (as above) and the IRES element (pCITE-4a(+) of Novagen as template, IRES forward=5′-TCCGGTTATTTTCCACCATATTGC-3′ and IRES reverse=5′-TTATCATCGTGTTTTTCAAAGG-3′) were one after the other cloned into the multiple cloning site of the pcDNA3.1(+) vector (Invitrogen). For such purpose the E1a-12S amplificate was introduced into the blunt-ended BamHI cleavage site by TA cloning. Subsequently, the plasmid E1a-12S was linearised in pcDNA3.1(+) with EcoRV, T ends added and the amplificate for the IRES element introduced by cloning. The thus obtained construct E1a-12S-IRES-pcDNA3.1(+) was linearised with NotI and the ends made blunt-ended; also the YB-1-EcoRI cleavage product from the plasmid pHVad2c CMV/S40+Yb-1 s (Stephan Bergmann) was made blunt-ended and introduced into the dephosphorylated vector E1A-12S-IRES-pcDNA3.1(+). Alternatively, the PCR amplificate for the open reading frame of protein IX may be introduced into the blunt-ended NotI cleavage site of the vector E1a-12S-IRES-pcDNA3.1(+) after addition of T ends, more specifically using the primers IX forward 5′-ATGAGCACCAACTCGTTTG-3′ and IX reverse 5′-GTTTTAAACCGCATTGGGAGG-3′.

The cassette E1A-12S-IRES-YB-1 or E1A-12S-IRES protein IX was excised with PmeI and cloned into the above-described plasmid pGL3-E2Late after removal of the luciferase gene with NcoI and XbaI and blunt-ending and dephosphorylation.

This cassette E2late-E1A-12S-IRES-YB-1 was excised with PvuI and ClaI, the ends made blunt-ended and can now alternatively be cloned into the EcoRII, BsiWI, DraI, MunI deleted E3a region (after removal of the open reading frames for E3 6.7 K and gp19 K, see above), or into the deletion of the E4ORF6, for example into the blunt-ended and dephosphorylated BfrI cleavage site.

The thus obtained construct is E3a/E2Late-E1a-12S-IRES-YB-1/ΔE3RGD-E4ΔORF6-pShuttle(-NdeI)-AdEASY or E3aΔE3RGD-E4ΔORF6/E2Late-E1a-12S-IRES-YB-1-pShuttle(-NdeI)-AdEASY.

Generation of the Rescue Plasmid E3a/E2Late-E1a-12S/ΔE3RGD-E4ΔORF6-pAdEASY or E3aΔE3RGD-E4ΔORF6/E2Late-E1a-12S-pAdEASY or E3a/E2Late-E1a-12S-IRES-YB-1/ΔE3RGD-E4ΔORF6-pAdEASY or E3aΔE3RGD-E4ΔORF6/E2Late-E1a-12S-IRES-YB-1-pAdEASY, Respectively

The E3aΔE3RGD-E4ΔORF6 region with the second expression cassette E2Late-E1a-12S or E2Late-E1a-12S-IRES-YB-1 in E3a or E4ΔORF6 was excised with SpeI and PacI from the corresponding pShuttle plasmid E3aΔE3RGD-E4ΔORF6-pShuttle(-NdeI)-AdEASY and cloned into the corresponding opened vector pAdEASY, which created the new rescue vector E3a/E2Late-E1a-12S/ΔE3RGD-E4ΔORF6-pAdEASY or E3aΔE3RGD-E4ΔORF6/E2Late-E1a-12S-pAdEASY or E3a/E2Late-E1a-12S-IRES-YB-1/ΔE3RGD-E4ΔORF6-pAdEASY or E3aΔE3RGD-E4ΔORF6/E2Late-E1a-12S-IRES-YB-1-pAdEASY, respectively.

E3aΔE3RGD-E4ΔORF6-pAdEASY contains the E3a region, an RGD motif and a deleted E4ORF6, as a second expression cassette either E2Late-E1a-12S or E2Late-E1a-12S-IRES-YB-1 are present in E3a or E4ΔORF6. This construct is the rescue plasmid for the introducing of further transgenes into the E1 region by a shuttle plasmid.

### Preparing the Transgene Cassette for the E1 Region

### Cloning of the E1B Region

For the E1B region the adenoviral genome was restricted with XbaI (position 1340 bp) and MunI (position 3925 bp) and the 2585 bp fragment was cloned into the pShuttle of AdEASY into the XbaI and MunI cloning sites which thus contains the complete E1B area (pShuttle/E1B).

Alternatively, the E1B region can be amplified by PCR with the primers E1B forward 5′-GTGTCTAGAGAATGCAATAGTAG-3′ and E1B reverse 5′-GTCAAAGAATCCAATTGTGC-3′ using the adenovirus type 5 wildtype DNA as template, be restricted with XbaI and MunI and cloned into the XbaI and MunI cleavage sites of the pShuttle of AdEASY.

Thus the pShuttle/E1B comprises the E1B promoter, the open reading frames for E1B19K, E1B55K and protein IX and the natural Poly-A part. The E1B promoter was removed by means of XbaI and HpaI, the ends of the vector made blunt-ended and replaced by the CMV promoter from pcDNA3.1(+) of Invitrogen which was cleaved with MluI and XhoI and the ends of which were also made blunt-ended. Alternatively, rather than the CMV promoter, an RSV promoter or a tumor-specific and viral promoters, respectively, for example the promoters mentioned in the patent, can control the expression of the E1B region.

Preparing the RSV Plasmid for the Preparation of the Cassette RSV-E4ORF6-polyA.

Plasmid pRc/RSV of Invitrogen was cleaved with XhoI, SpeI and XbaI. The thus resulting 2810 bp and 278 bp fragments were religated such that by doing so the F1 origin and the neomycine resistance gene (oNeo) were removed.

The thus obtained vector pRc/RSV (oNeo) comprises only one BamHI cleavage site into which the open reading frame for E4ORF6 from plasmid CGN from Dobbelstein was cloned. Alternatively, the amplificate of a PCR using the primers E4ORF6-forward 5′-ATGACTACGTCCGGCGTTCC-3′ and E4ORF6-reverse 5′-CTACATGGGGGTAGAGTC-3′ can be introduced into the EcoRV cleavage site of the vector pRc/RSV (oNeo) after adding the T ends (TA cloning). Alternatively, rather than the RSV promoter (by excision using MliI and HindIII), a CMV promoter (obtained from the pcDNA3.1(+) by excision with MluI and HindIII) or a tumor-specific and viral promoter, respectively, for example the promoters described in the patent, can control the expression of E4orf6.

The cassette RSV-E4ORF6-polyA (the bovine growth hormone polyadenylation signal is derived from plasmid pRC/RSV) was cleaved with MunI, the ends made blunt-ended and further excised with XhoI from the plasmid. The expression cassette was subsequently cloned into the vector pShuttle/E1B which had been cleaved by NotI, made blunt-ended and subsequently cleaved with XhoI. From this vector RSV-E4ORF6-polyA/E1B-pShuttle-AdEASY was obtained.

Introducing the Transgenic Cassette into the Rescue Vector

The vector RSV-E4ORF6-polyA/E1B-pShuttle-AdEASY for the E1-Bereich was linearised using Bst1107I and MroI and introduced together with the rescue plasmid (see above) into (EC) bacteria by means of electroporation. The adenoviral plasmid RSV-E4ORF6-polyA/E1B-E3a/E2Late-E1a-12S/ΔE3RGD-E4ΔORF6-pAdEASY was generated (or in a corresponding manner with the other above mentioned rescue vector variants) by homologous recombination which resulted in virus production after transfection in HEK293 cells.

It is within the present invention and obvious for a person skilled in the art in the light of the present disclosure that other systems, such as, for example, pAdenoX-System of Clontech/BD Biosciences or the system of Microbix may be used for the manufacture of the adenoviruses, preferably recombinant adenoviruses, according to the invention, in particular those which contain the above expression cassettes individually and/or in any combination.

The features disclosed in the preceding description, the claims as well as the figures may be individually or in any combination essential for the practising of the invention in its various embodiments.

1.-83. (canceled)

84. A virus, preferably an adenovirus, characterised in that the virus comprises:
a lacking functional wildtype E1 region, whereby the lacking functional wildtype E1 region is protein IX-minus, and
a transporter for the transport of YB-1 into the nucleus of a cell which is infected with the virus, and
that the virus comprises a nucleic acid coding for protein IX and expresses protein IX.

85. The virus according to claim 84, characterised in that the lacking functional wildtype E1A region is E1A-minus.

86. The virus according to claim 84, characterised in that the lacking functional wildtype E1 region is E1B-minus.

87. The virus according to claim 86, characterised in that the lacking wildtype E1 region is E1B55K-minus and/or E1B19K-minus.

88. The virus according to claim 87, characterised in that the transporter is a transporter provided by the virus.

89. The virus according to claim 88, characterised in that the transporter is a viral transporter.

90. The virus according to claim 84, characterised in that the transporter comprises protein E4orf6.

91. The virus according to claim 91, characterised in that the transporter comprises protein E1B55K.

92. The virus according to claim 88, characterised in that the transporter comprises a complex of E4orf4 and E1B55K.

93. The virus according to claim 84, characterised in that the transporter is coded by a nucleic acid, whereby the nucleic acid is under the control of a promoter.

94. The virus according to claim 93, characterised in that the transporter is a complex of at least two factors and whereby each factor is coded by a nucleic acid, whereby both nucleic acids are controlled by a shared promoter.

95. The virus according to claim 94, characterised in that both nucleic acid are connected through an element which controls the expression strength, whereby the element is preferably selected from the group comprising IRES.

96. The virus according to claim 93, characterised in that the transporter is a complex of at least two factors and whereby each factor is coated by a nucleic acid, whereby both nucleic acids are controlled by a proprietary promoter.

97. The virus according to claim 93, characterised in that the promoter is different from the E4 promoter, in particular the adenoviral E4 promoter and is different from the E1B promoter, in particular the adenoviral E1B promoter.

98. The virus according to claim 93, characterised in that the promoter is selected from the group comprising tissue-specific promoters, tumor-specific promoters, the CMV-promoter, viral promoters and particularly adenoviral promoters under the proviso that these are different from the E4 promoter, the E1B promoter and preferably also different from the E2-late promoter.

99. The virus according to claim 84, characterised in that the nucleic acid coding for the transporter has a 3′-UTR at the 3′ end of E1B55K.

100. The virus according to claim 84, characterised in that if the lacking wildtype E1 region is EB55K-positive, the nucleic acid coding for the transporter does not comprise an E1B55K coding nucleic acid.

101. The virus according to claim 84, characterised in that the nucleic acid coding for the transporter codes for E1B55K and E1B19K.

102. The virus according to claim 101, characterised in that the nucleic acid coding for the transporter codes for protein IX.

103. The virus according to claim 101, characterised in that the nucleic acid coding for the E1B55K and E1B19K is under the control of a promoter.

104. The virus according to claim 101, characterised in that the nucleic acid coding for the E1B55K and/or E1B19K and/or protein IX is under the control of a promoter, whereby the promoter is different from an E1A-dependent promoter.

105. The virus according to claim 84, characterised in that the lacking functional wildtype E1 region is E1A13S-minus and/or E1A12S-minus.

106. The virus according to claim 84, characterised in that the lacking functional wildtype E1 region is E1A13S-minus.

107. The virus according to claim 84, characterised in that preferably the lacking wildtype E1 region is E1A13S-minus and E1A12-minus, whereby the virus comprises a nucleic acid coding for the E1A12S protein, whereby the nucleic acid is preferably a heterologous nucleic acid.

108. The virus according to claim 107, characterised in that the nucleic acid coding for the E1A12S protein is under the control of a promoter, whereby the promoter is preferably a YB-1 dependent promoter and more preferably selected from the group comprising the adenoviral E2-late promoter, the MDR-promoter and the DNA polymerase alpha promoter.

109. The virus according to claim 108, characterised in that the nucleic acid(s) coding for the transporter code for E4orf6 and E1B55K.

110. The virus according to claim 108, characterised in that the virus comprises a nucleic acid coding for protein IX, whereby preferably the nucleic acid coding for E1A12S and the nucleic acid coding for protein IX are under the control of a shared promoter, whereby more preferably both nucleic acids are linked to each other through an expression regulating element, whereby the element is more preferably selected from the group comprising IRES.

111. The virus according to claim 107, characterised in that the nucleic acid coding for the E1A12S protein and the nucleic acid coding for the protein IX are each under the control of a promoter, whereby the promoter is preferably the same promoter.

112. The virus according to claim 110, characterised in that the promoter is a YB-1 dependent promoter, which is preferably selected from the group comprising the adenoviral E2-late promoter, the MDR promoter and the DNA polymerase-alpha promoter.

113. The virus according to claim 107, characterised in that the virus comprises a YB-1 coding nucleic acid.

114. The virus according to claim 113, characterised in that the nucleic acid coding for the E1A12S protein and the nucleic acid coding for the YB-1 are under the control of a shared promoter, whereby preferably both nucleic acids are linked to each other by an expression regulating element, whereby the element is preferably selected from the group comprising IRES.

115. The virus according to claim 113, characterised in that the nucleic acid coding for YB-1 and the nucleic acid coding for E1A12S protein are each under the control of a promoter, whereby the promoter is preferably the same promoter.

116. The virus according to claim 113, characterised in that the promoter is a YB-1 dependent promoter which is preferably selected from the group comprising the adenoviral E2-late promoter, the MDR promoter and the DNA polymerase-alpha promoter.

117. The virus according to claim 107, characterised in that the nucleic acid coding for E1A12S is cloned into the E3 region or E4 region.

118. The virus according to claim 107, characterised in that the nucleic acid coding for E1A12S and the nucleic acid coding for the protein IX or the nucleic acid coding for the YB-1 are cloned into the E3 region or the E4 region.

119. The virus according to claim 114, characterised in that the expression of the nucleic acid coding for protein IX is controlled by a promoter different from E1B via E1B19K or via E12AS.

120. The virus according to claim 84, characterised in that the virus comprises at least one transgene which is preferably cloned into the E3 region.

121. The virus according to claim 120, characterised in that the virus comprises at least one transgene which is preferably cloned into the E4 region.

122. The virus according to claim 84 comprising a nucleic acid coding for the RGD motif, whereby the RGD motif is preferably cloned into the HI-loop domain of the fibre knob.

123. The virus according to claim 84, further comprising MLP genes and/or E2A genes and E2B genes and/or E3 genes and/or E4 genes.

124. The virus according to claim 84, characterised in that the virus is replication deficient in cells which do not contain YB-1 in the nucleus.

125. The virus according to claim 84, characterised in that the virus can replicate in cells which have YB-1 in the nucleus, in particular have YB-1 in the nucleus independent of the cell cycle.

126. The virus according to claim 84, characterised in that the virus is replication deficient in cells where or in which YB-1 is deregulated.

127. The virus according to claim 84, characterised in that the virus is capable of replicating in tumor cells, preferably tumor cells which are resistant against cytostatics and/or radiation.

128. The virus according to claim 127, characterised in that the cells are multiple-drug resistant.

129. A nucleic acid coding for a virus according to claim 84 or a part thereof.

130. Use of a virus according to claim 84 or a nucleic acid according to claim 129 or a vector comprising the same or a replication system comprising such nucleic acid or a part thereof, for the manufacture of a medicament.

131. Use of a virus according to claim 84 or a nucleic acid according to claim 129 for replication in cells, whereby the cells contain YB-1 in the nucleus, preferably contain YB-1 in the nucleus independent of the cell cycle, or the cells contain deregulated YB-1 or that the cells are tumor cells, preferably tumor cells which are resistant against cytostatics and/or radiation.

132. Use according to claim 131, characterised in that the cells contain YB-1 in the nucleus after or due to a measure which is applied to the cell or has been applied to the cell and is selected from the group comprising radiation, application of cytostatics and hyperthermia.

133. Use according to claim 131, characterised in that the medicament is for the treatment of tumors and/or cancer(s) and/or for the restoration of sensitivity of cells to cytostatics and/or radiation, whereby preferably the cells are tumor cells which are resistant against cytostatics and/or radiation.

134. Use according to claim 133, characterised in that at least one part of the cells forming the tumor are cells which have YB-1 in the nucleus, preferably contain YB-1 in the nucleus independent of the cell cycle, or that at least a part of the cells forming the tumor have deregulated YB-1 or at least a part of the cells forming the tumor are tumor cells, more preferably tumor cells which are resistant against cytostatics and/or radiation.

135. Use according to claim 134, characterised in that the cells, particularly the cells forming the tumor or parts thereof, are resistant, in particular multi-resistant against drugs, preferably antitumor agents and more preferably cytostatics.

136. Use according to claim 135, characterised in that the cells show an expression, more preferably an overexpression of the membrane bound transport protein P-glycoprotein.

137. Use according to claim 131, characterised in that the cells have YB-1 in the nucleus, and particularly that the cells forming the tumor or part thereof have YB-1 in the nucleus.

138. Use according to claim 131 characterised in that the tumor contains YB-1 in the nucleus after induction of the transport of YB-1 into the nucleus.

139. Use according to claim 139, characterised in that the transport of YB-1 into the nucleus is triggered by at least one measure which is selected from the group comprising radiation, application of cytostatics and hyperthermia.

140. Use according to claim 139, characterised in that the measure is applied to a cell, an organ or an organism.

141. Use of a virus replication system, particularly an adenoviral replication system, comprising a nucleic acid which codes for a virus, particularly an adenovirus, according to claim 84 or a part thereof, and comprising a nucleic acid of a helper virus, whereby the nucleic acid of the helper virus comprises a nucleic acid sequence which codes for YB-1, and optionally complements the virus, preferably for the manufacture of a medicament, more preferably for the treatment of tumors and/or cancer(s) and/or for restoration of the sensitivity of cells to cytostatics and/or radiation, whereby the cells are preferably tumor cells which are resistant against cytostatics and/or radiation.

142. Use of a viral replication system according to claim 141, characterised in that the viral nucleic acid, preferably the adenoviral nucleic acid and/or the nucleic acid of the helper virus are present as replicable vector.

143. Use of a nucleic acid coding for a virus, preferably an adenovirus according to claim 84 for the manufacture of a medicament, preferably for the manufacture of a medicament for the treatment of tumors and/or for restoration of sensitivity of cells to cytostatics and/or radiation, whereby the cells are preferably tumor cells which are resistant against cytostatics and/or radiation.

144. Use according to claim 143, characterised in the cells, and particularly the cells forming the tumor or parts thereof, are resistant, in particular multiple-resistant against drugs, preferably antitumor agents and more preferably cytostatics.

145. A vector comprising a nucleic acid according to claim 129.

146. Use of an agent interacting with YB-1 for the characterisation of cells, cells of a tumor tissue or patients, in order to determine whether such cells, cells of a tumor tissue or patients can/should be contacted and/or treated with a virus according to claim 84 or a nucleic acid according to claim 129.

147. Use according to claim 146, characterised in that the agent is selected from the group comprising antibodies, high affinity binding peptides, antikalines, aptamers, aptazymes and spiegelmers.

148. A pharmaceutical composition comprising a virus according to claim 84, or a nucleic acid according to claim 129 or a viral replication system as described in claim 141.

149. The pharmaceutical composition according to claim 148, whereby the composition comprises at least one further pharmaceutically active agent.

150. The pharmaceutical composition according to claim 149, whereby the pharmaceutically active agent is selected from the group comprising cytokines, metalloproteinase inhibitors, angiogenesis inhibitors, cytostatics, cell cycle inhibitors, proteosome inhibitors, recombinant antibodies, inhibitors of the signal transduction cascade and protein kinases.

151. The pharmaceutical composition according to claim 148, characterised in that the composition comprises a combination of at least two compounds, whereby preferably any compound is each and independently selected from the group comprising cytostatics.

152. The pharmaceutical composition according to claim 151, characterised in that at least two of the compounds target different target molecules.

153. The pharmaceutical composition according to claim 152, characterised in that at least two of the compounds are active through different modes of action.

154. The pharmaceutical composition according to claim 151, characterised in that at least one compound increases the infectability of a cell in which the virus is replicating.

155. The pharmaceutical composition according to claim 151, characterised in that at least one compound influences the availability of a compound in the cell, preferably increases the availability of the compound, whereby the compound mediates the uptake of the virus in one or the cell, preferably the one in which the virus replicates.

156. The pharmaceutical composition according to claim 151, characterised in that at least one of the compound mediates the transport of YB-1 into the nucleus, preferably increases the same.

157. The pharmaceutical composition according to claim 151, characterised in that at least one compound is a histone deacylase inhibitor.

158. The pharmaceutical composition according to claim 157, characterised in that the histone deacylase inhibitor is selected from the group comprising trichostatine A, FR 901228, MS-27-275, NVP-LAQ824, PXD101, apicidine and scriptaid.

159. The pharmaceutical composition according to claim 151, characterised in that at least one compound is selected from the group comprising trichostatine A, FR 901228, MS-27-275, NVP-LAQ824, PXD101, apicidine and scriptaid.

160. The pharmaceutical composition according to claim 151, characterised in that at least one compound is a topoisomerase inhibitor.

161. The pharmaceutical composition according to claim 160, characterised in that the topoisomerase inhibitor is selected from the group comprising camptothecin, irinotecan, toptecan, DX-895If, SN-38, 9-aminocamptothecin, 9-nitrocamptothecin, daunorubicin and etoposid.

162. The pharmaceutical composition according to claim 161, characterised in that the composition comprises trichostatine A and irinotecan.

163. The pharmaceutical composition according to claim 148, characterised in that the virus, in particular the virus according to claim 84 is separated from one or both or all of the at least two compounds.

164. The pharmaceutical composition according to claim 163, characterised in that at least one unit dose of the virus is separated from at least one unit dose of the or all further pharmaceutically active compound(s) or from one or the at least two compounds.

165. A kit comprising a virus according to claim 163, and at least two pharmaceutically active agents, whereby each pharmaceutically active agent is individually and independently selected from the group comprising cytostatics.
